(19) United States
(12) Patent Application Publication
(10) Pub. No.: US 2026/0291522 A1
(43) Pub. Date: Sep. 24, 2026

(54) Iterative Block Decoding Using Soft Information from Multiple Oligo Copies in DNA Data Storage

(71) Applicant: Western Digital Technologies, Inc., San Jose, CA (US)

(72) Inventors: Iouri Oboukhov, Rochester, MN (US); Richard Galbraith, Rochester, MN (US); Niranjay Ravindran, Rochester, MN (US); Austin Striegel, Rochester, MN (US)

(21) Appl. No.: 19/082,721

(22) Filed: Mar. 18, 2025

(51) Int. Cl. H03M 13/15; G16B 30/20; G16B 50/50

(52) U.S. Cl. CPC H03M 13/1515; G16B 30/20; G16B 50/50

(57) ABSTRACT

Example systems and methods for using soft information from oligo copies in a set of oligos for iterative decoding and sequencing in DNA data storage are described. Read data from an oligo pool may include a variable number of copies of each oligo in the set. During decoding, a correlation matrix may be constructed for each copy of an oligo and traversed to calculate a most likely path that generates a set of soft information for each element in the correlation matrix. The sets of soft information from multiple copies may be used to iteratively decode each oligo, then aggregated into a larger aggregate decoding block for a second stage of iterative decoding. Failure to decode the aggregate decoding block may trigger additional amplification and sequencing to increase the number of oligo copies for a next round of decoding.

### TECHNICAL FIELD

The present disclosure relates to deoxyribonucleic acid (DNA) data storage. In particular, the present disclosure relates to error correction for data stored as a set of synthetic DNA oligos.

### BACKGROUND

DNA is a promising technology for information storage. It has potential for ultra-dense three-dimensional (3D) storage with high storage capacity and longevity. Currently, the technology of DNA synthesis provides tools for synthesis and manipulation of relatively short synthetic DNA chains (oligos). For example, some oligos may include 40 to 350 bases encoding twice that number of bits in configurations that use bit symbols mapped to the four DNA nucleotides or sequences thereof. Due to the relatively short payload capacity of oligos, Reed-Solomon error correction codes have been applied to individual oligos.

There is a need for technology that applies more efficient error correction to DNA data storage and retrieval.

### BRIEF DESCRIPTION OF THE DRAWINGS

The techniques introduced herein are illustrated by way of example, and not by way of limitation, in the figures of the accompanying drawings in which like reference numerals are used to refer to similar elements.

FIG. 1A is a block diagram of a prior art DNA data storage process.

FIG. 1B is a block diagram of a prior art DNA data storage decoding process for oligos encoded with binary data.

FIG. 2 is a block diagram of an example encoding system and example decoding system for DNA data storage using soft information for iterative block decoding.

FIGS. 3A and 3B are diagrams of oligo processing to determine the reference marks in oligo read data.

FIG. 4 is a block diagram of an oligo data processing system using soft information for iterative block decoding.

FIGS. 5A, 5B, and 5C are diagrams of using soft information from a correlation matrix to determine consensus probabilities for each base pair.

FIG. 5D is a diagram of using hard decisions from multiple copies to determine soft information based on consensus probabilities for each base pair.

FIGS. 6A, 6B, and 6C are example methods for decoding user data using consensus of soft information and block decoding, such as using the decoding systems of FIGS. 2 and 4.

FIG. 7 is an example method for encoding oligos, such as using the encoding system of FIG. 2.

FIG. 8 is an example method for determining probabilities of reference mark shifts and associated soft information using a Viterbi algorithm, such as using the decoding systems of FIGS. 2 and 4.

FIG. 9 is an example method for iteratively decoding aggregate blocks based on additional read data from amplification and sequencing.

### SUMMARY

Various aspects for using soft information from multiple copies of oligos to iteratively decode data based on aggregate decode blocks stored in an oligo pool for DNA data storage are described.

One general aspect includes a system that includes at least one decoder circuit configured to, alone or in combination: receive a first set of read data determined from sequencing a set of oligos that includes first numbers of copies of each oligo and corresponds to an oligo pool configured to store a data unit; determine, from the first set of read data for each copy of each oligo, a correlation matrix of possible base pair offsets for that copy of the oligo; determine, for each copy of each oligo, a most likely path through the correlation matrix for that copy of that oligo, where calculation of the most likely path generates a set of soft information for each element in the correlation matrix for that copy of that oligo; aggregate, for each oligo, an aggregate set of soft information for each base pair along a length of that oligo; decode, using a first iterative decoder, payload data in each oligo using the aggregate set of soft information for that oligo to determine decoded payload data for that oligo; map decoded payload data for each oligo to data positions in an aggregate decoding block; decode, using a second iterative decoder, the aggregate decoding block to determine a decoded aggregate decoding block for the data unit; and determine, based on the decoded aggregate decoding block, whether the data unit can be successfully decoded.

Implementations may include one or more of the following features. The at least one decoder circuit may be further configured to, alone or in combination, responsive to determining that the data unit cannot be successfully decoded from the decoded aggregate decoding block: selectively initiate further sequencing of the oligo pool to generate a next set of read data; receive the next set of read data that includes a second number of copies of each oligo; determine a prior set of read data based on the first set of read data and any subsequent iterations of selectively initiating further sequencing; initiate, from the next set of read data and for each copy of each oligo, determining the correlation matrices, most likely paths, and sets of soft information from the next set of read data; aggregate, for each oligo, an updated aggregate set of soft information using the sets of soft information from the prior set of read data and the next set of read data; decode, using the first iterative decoder, the payload data in each oligo using the updated aggregate set of soft information for that oligo to determine updated decoded payload data for that oligo; update the decoded payload data for each oligo in the aggregate decoding block; decode, using the second iterative decoder, the aggregate decoding block to determine an updated decoded aggregate decoding block; and determine, based on the updated decoded aggregate decoding block, whether the data unit can be successfully decoded. The at least one decoder circuit may be further configured to, alone or in combination, iteratively execute, until the data unit is successfully decoded: the selectively initiating further sequencing; the receiving the next set of read data; the determining the prior set of read data; the initiating determining the correlation matrices, most likely paths, and sets of soft information; the aggregating the updated aggregate set of soft information; the decoding the payload data in each oligo; the updating the decoded payload data for each oligo in the aggregate decoding block; the decoding the aggregate decoding block; and the determining whether the data unit can be successfully decoded. The at least one decoder circuit may be further configured to, alone or in combination, responsive to determining that the data unit cannot be successfully decoded from the decoded aggregate decoding block: determine a target number of copies of each oligo for selectively initiating further sequencing of the oligo pool to generate a next set of read data; and provide the target number of copies to an amplification and sequencing system for the oligo pool. The at least one decoder circuit may be further configured to, alone or in combination, responsive to determining that the data unit cannot be successfully decoded from the decoded aggregate decoding block: determine an error rate for the first set of read data; and determine an error correction capability for the second iterative decoder. Determining the target number of copies of each oligo may be based on the error rate for the first set of read data and the error correction capability for the second iterative decoder. The at least one decoder circuit may be further configured to, alone or in combination, prior to decoding the payload data in each oligo: determine, for each oligo, most likely positions for a series of reference marks configured with a reference pattern and a predetermined interval of base pairs; correct, for each oligo, insertions and deletions to restore the predetermined interval of base pairs for the series of reference marks; and remove, for each oligo, the reference marks from remaining base pairs in that oligo to determine the payload data to be decoded. The at least one decoder circuit may be further configured to, alone or in combination: determine, from the first set of read data for each copy of each oligo, a convolutional matrix for that copy of that oligo, where each column of the convolutional matrix corresponds to a base pair offset of the read data for that copy of the oligo; determine, for each copy of each oligo, at least one reference matrix based on at least one possible reference pattern for that oligo; and compare, for each copy of each oligo, the convolutional matrix to the at least one reference matrix to determine the correlation matrix of possible base pair offsets for that copy of the oligo. The system may include a data storage device configured with a read channel block size, where: the at least one decoder circuit is further configured to, alone or in combination, store, prior to decoding the aggregate decoding block, the aggregate decoding block to a non-volatile storage medium of the data storage device; and the read channel block size is at least as large as a block size of the aggregate decoding block. Mapping the decoded payload data for each oligo to data positions in the aggregate decoding block may include: determining, for each oligo in the set of oligos, an oligo address for that oligo configured to indicate a position of the decoded payload data of that oligo relative to positions of the decoded payload data of other oligos; and ordering, based on the oligo addresses for each oligo in the set of oligos, the decoded payload data in the aggregate decoding block. Determining whether the data unit can be successfully decoded may include: executing a cyclic redundancy check on the decoded aggregate decoding block; outputting, responsive to the cyclic redundancy check being successful, the decoded aggregate decoding block for recovering and storing the data unit; and initiating, responsive to the cyclic redundancy check not being successful, further amplification and replication of the oligo pool.

Another general aspect includes a method that includes: receiving a first set of read data determined from sequencing a set of oligos that includes first numbers of copies of each oligo and corresponds to an oligo pool configured to store a data unit; determining, from the first set of read data for each copy of each oligo, a correlation matrix of possible base pair offsets for that copy of the oligo; determining, for each copy of each oligo, a most likely path through the correlation matrix for that copy of that oligo, where calculation of the most likely path generates a set of soft information for each element in the correlation matrix for that copy of that oligo; aggregating, for each oligo, an aggregate set of soft information for each base pair along a length of that oligo; decoding, using a first iterative decoder, payload data in each oligo using the aggregate set of soft information for that oligo to determine decoded payload data for that oligo; mapping decoded payload data for each oligo to data positions in an aggregate decoding block; decoding, using a second iterative decoder, the aggregate decoding block to determine a decoded aggregate decoding block for the data unit; and determining, based on the decoded aggregate decoding block, whether the data unit can be successfully decoded.

Implementations may include one or more of the following features. The method may include, responsive to determining that the data unit cannot be successfully decoded from the decoded aggregate decoding block: selectively initiating further sequencing of the oligo pool to generate a next set of read data; receiving the next set of read data that includes a second number of copies of each oligo; determining a prior set of read data based on the first set of read data and any subsequent iterations of selectively initiating further sequencing; initiating, from the next set of read data and for each copy of each oligo, determining the correlation matrices, most likely paths, and sets of soft information from the next set of read data; aggregating, for each oligo, an updated aggregate set of soft information using the sets of soft information from the prior set of read data and the next set of read data; decoding, using the first iterative decoder, the payload data in each oligo using the updated aggregate set of soft information for that oligo to determine updated decoded payload data for that oligo; updating the decoded payload data for each oligo in the aggregate decoding block; decoding, using the second iterative decoder, the aggregate decoding block to determine an updated decoded aggregate decoding block; and determining, based on the updated decoded aggregate decoding block, whether the data unit can be successfully decoded. The method may include iteratively executing, until the data unit is successfully decoded: the selectively initiating further sequencing; the receiving the next set of read data; the determining the prior set of read data; the initiating determining the correlation matrices, most likely paths, and sets of soft information; the aggregating the updated aggregate set of soft information; the decoding the payload data in each oligo; the updating the decoded payload data for each oligo in the aggregate decoding block; the decoding the aggregate decoding block; and the determining whether the data unit can be successfully decoded. The method may include, responsive to determining that the data unit cannot be successfully decoded from the decoded aggregate decoding block: determining a target number of copies of each oligo for selectively initiating further sequencing of the oligo pool to generate a next set of read data; and providing the target number of copies to an amplification and sequencing system for the oligo pool. The method may include, responsive to determining that the data unit cannot be successfully decoded from the decoded aggregate decoding block: determining an error rate for the first set of read data; and determining an error correction capability for the second iterative decoder, where determining the target number of copies of each oligo is based on the error rate for the first set of read data and the error correction capability for the second iterative decoder. The method may include, prior to decoding the payload data in each oligo: determining, for each oligo, most likely positions for a series of reference marks configured with a reference pattern and a predetermined interval of base pairs; correcting, for each oligo, insertions and deletions to restore the predetermined interval of base pairs for the series of reference marks; and removing, for each oligo, the reference marks from remaining base pairs in that oligo to determine the payload data to be decoded. The method may include: determining, from the first set of read data for each copy of each oligo, a convolutional matrix for that copy of that oligo, where each column of the convolutional matrix corresponds to a base pair offset of the read data for that copy of the oligo; determining, for each copy of each oligo, at least one reference matrix based on at least one possible reference pattern for that oligo; and comparing, for each copy of each oligo, the convolutional matrix to the at least one reference matrix to determine the correlation matrix of possible base pair offsets for that copy of the oligo. Mapping the decoded payload data for each oligo to data positions in the aggregate decoding block may include: determining, for each oligo in the set of oligos, an oligo address for that oligo configured to indicate a position of the decoded payload data of that oligo relative to positions of the decoded payload data of other oligos; and ordering, based on the oligo addresses for each oligo in the set of oligos, the decoded payload data in the aggregate decoding block. Determining whether the data unit can be successfully decoded may include: executing a cyclic redundancy check on the decoded aggregate decoding block; outputting, responsive to the cyclic redundancy check being successful, the decoded aggregate decoding block for recovering and storing the data unit; and initiating, responsive to the cyclic redundancy check not being successful, further amplification and replication of the oligo pool.

Still another general aspect includes a system that includes at least one processor; at least one memory; means for receiving a first set of read data determined from sequencing a set of oligos that includes first numbers of copies of each oligo and corresponds to an oligo pool configured to store a data unit; means for determining, from the first set of read data for each copy of each oligo, a correlation matrix of possible base pair offsets for that copy of the oligo; means for determining, for each copy of each oligo, a most likely path through the correlation matrix for that copy of that oligo, where calculation of the most likely path generates a set of soft information for each element in the correlation matrix for that copy of that oligo; means for aggregating, for each oligo, an aggregate set of soft information for each base pair along a length of that oligo; means for decoding, using a first iterative decoder, payload data in each oligo using the aggregate set of soft information for that oligo to determine decoded payload data for that oligo; means for mapping decoded payload data for each oligo to data positions in an aggregate decoding block; means for decoding, using a second iterative decoder, the aggregate decoding block to determine a decoded aggregate decoding block for the data unit; and means for determining, based on the decoded aggregate decoding block, whether the data unit can be successfully decoded.

The present disclosure describes various aspects of innovative technology capable of applying soft information from multiple copies of an oligo from a DNA oligo pool to aggregate and decode blocks of user data stored in the DNA oligo pool. The iterative decoding provided by the technology may be applicable to a variety of computer systems used to store or retrieve data stored as a set of oligos in a DNA storage medium. The configuration may be applied to a variety of DNA synthesis and sequencing technologies to generate write data for storage as base pairs and process read data read from those base pairs. The novel technology described herein includes a number of innovative technical features and advantages over prior solutions, including, but not limited to, improved data recovery based on elimination of insertion and deletion errors, more accurate consensus decisions for correcting user data, and multiple levels of error correction dynamically matched to amplification and synthesis activities for the oligo pool.

### DETAILED DESCRIPTION

Prior methods for decoding data units encoded in DNA oligo pools have used consensus decision-making for base pairs, generally based on a high volume of oligo copies. For example, an oligo pool for a 512 kilobyte (KB) data block may include more than 50,000 unique oligos to encode the user data and associated redundancy data. Replication and amplification of those oligos to reliably use consensus-based decoding may represent significant overhead for both the DNA sequencing and subsequent data processing. For example, if reliable consensus processing requires at least 10 copies, then the amplification will need to target a much higher number of copies to achieve a desired distribution of randomly generated copies. Assuming a three sigma distribution (in each direction) that has at least 10 copies for 99.9% of the oligos, the mean number of copies targeted could be 100 or more and require replication, sequencing, and data processing of millions or billions of oligos.

However, improved data processing and error correction may reduce the number of copies for reliable consensus-based decoding stages that leverage multiple copies while not requiring the high number of copies. In addition, these improved consensus-based techniques may use the number of copies of each oligo in the read data from the pool to determine the consensus function used. More specifically, rather than direct consensus “voting” of individual base pair values across the multiple copies of the oligo, soft information from probabilistic determination of base pair values may be aggregated and compared to generate more accurate consensus averages. For example, the aggregation of Viterbi probabilities from a correlation matrix may provide more reliable consensus averages from far fewer copies and may even support some form of consensus improvement on as few as two copies. These soft information consensus techniques may enable smaller read data sets that target a mean of 3-7 copies and less than 10 copies for the majority of the oligos in the set.

Further, because the number of copies and fidelity of those copies are both variable, multiple levels of data encoding and iterative decoding may be selectively applied depending on the resulting error rates from successive numbers of copies. For example, the first level of data encoding may utilize consensus of soft information to generate best copies of oligo data that are then aggregated into a hyperblock for another layer of decoding based on additional redundancy data and error correction code (ECC) encoding. This aggregate block layer may be verified and unsuccessful decoding may trigger additional generation of read data having additional copies of the oligos in the data pool. Calibrating the target number of copies to aggregate error rates and error correction capabilities of the hyperblock ECC may allow the system to iteratively generate read data through amplification and synthesis of the oligo pool until the hyperblock is successfully decoded or a maximum resource threshold is reached.

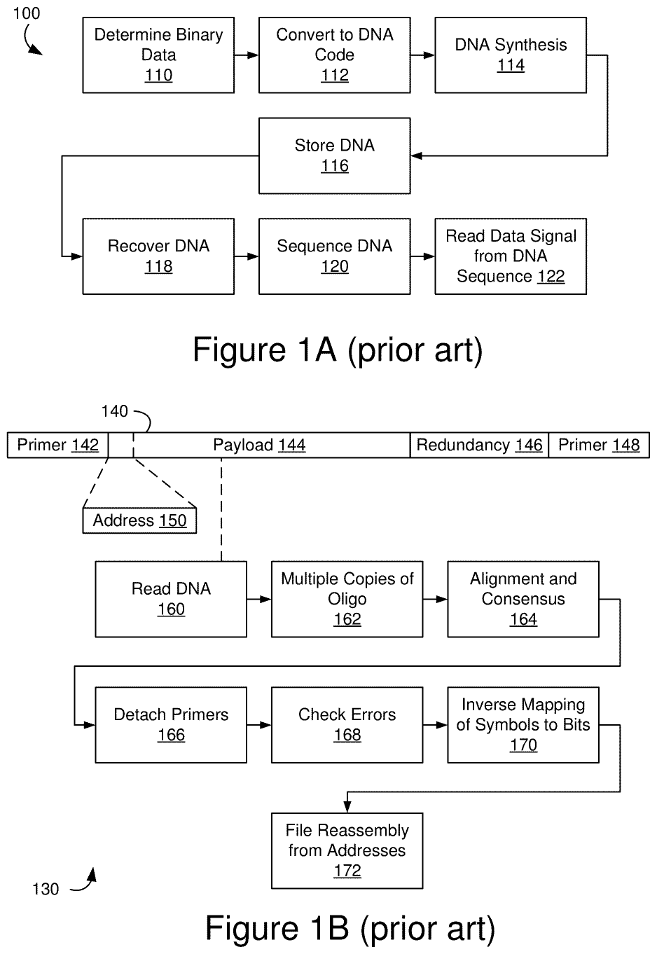

Novel data storage technology is being developed to use synthesized DNA encoded with binary data for long-term data storage. While current approaches may be limited by the time it takes to synthesize and sequence DNA, the speed of those systems is improving, and the density and durability of DNA as a data storage medium is compelling. In an example configuration in FIG. 1A, a method 100 may be used to store and recover binary data from synthetic DNA.

At block 110, binary data for storage to the DNA medium may be determined. For example, any conventional computer data source may be targeted for storage in a DNA medium, such as data files, databases, data objects, software code, etc. Due to the high storage density and durability of DNA media, the data targeted for storage may include very large data stores having archival value, such as collections of images, videos, scientific data, software, enterprise data, and other archival data. These conventional data sources may include data units for storage, such as data blocks of a particular size, such as 512 kilobyte (KB), 1 megabyte (MB), 2 MB, or 4 MB blocks.

At block 112, the binary data may be converted to DNA code. For example, a conventional computer data object or data file may be encoded according to a DNA symbol index, such as: A or T=1 and C or G=0; A=00, T=01, C=10, and G=11; or a more complex DNA symbol index mapping sequences of DNA bases to predetermined binary data patterns. In some configurations, prior to conversion to DNA code, the source data may be encoded according to an oligo-length format that includes addressing and redundancy data for use in recovering and reconstructing the source data during the retrieval process.

At block 114, DNA may be synthesized to embody the DNA code determined at block 112. For example, the DNA code may be used as a template for generating a plurality of synthetic DNA oligos embodying that DNA code using various DNA synthesis techniques. In some configurations, a large data unit is broken into segments matching a payload capacity of the oligo length being used, and each segment is synthesized in a corresponding DNA oligo. In some configurations, solid-phase DNA synthesis may be used to create the desired oligos. For example, each desired oligo may be built on a solid support matrix one base at a time to match the desired DNA sequence, such as using phosphoramidite synthesis chemistry in a four-step chain elongation cycle. In some configurations, column-based or microarray-based oligo synthesizers may be used.

At block 116, the DNA medium may be stored. For example, the resulting set of DNA oligos for the data unit may be placed in a fluid or solid carrier medium. The resulting DNA medium of the set of oligos and their carrier may then be stored for any length of time with a high-level of stability (e.g., DNA that is thousands of years old had been successfully sequenced). In some configurations, the DNA medium may include wells of related DNA oligos suspended in carrier fluid or a set of DNA oligos in a solid matrix that can themselves be stored or attached to another object. A set of DNA oligos stored in a binding medium may be referred to as a DNA storage medium for an oligo pool. The DNA oligos in the pool may relate to one or more binary data units comprised of user data (the data to be stored prior to encoding and addition of syntactic data, such as headers, addresses, reference marks, etc.).

At block 118, the DNA oligos may be recovered from the stored medium. For example, the oligos may be separated from the carrier fluid or solid matrix for processing. The resulting set of DNA oligos may be transferred to a new solution for the sequencing process or may be stored in a solution capable of receiving the other polymerase chain reaction (PCR) reagents.

At block 120, the DNA oligos may be sequenced and read into a DNA data signal corresponding to the sequence of bases in the oligo. For example, the set of oligos may be processed through PCR to amplify the number of copies of the oligos from the stored set of oligos. In some configurations, PCR amplification may result in a variable number of copies of each oligo.

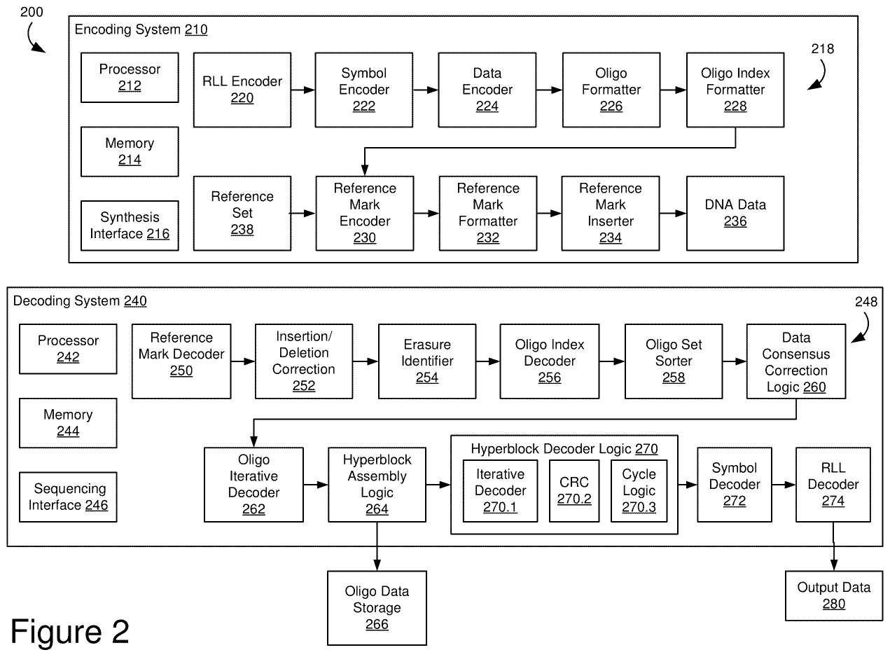

At block 122, a data signal may be read from the sequenced DNA oligos. For example, the sequenced oligos may be passed through a nanopore reader to generate an electrical signal corresponding to the sequence of bases. In some configurations, each oligo may be passed through a nanopore, and a voltage across the nanopore may generate a differential signal with magnitudes corresponding to the different resistances of the bases. The analog DNA data signal may then be converted back to digital data based on one or more decoding steps, as further described with regard to a method 130 in FIG. 1B. Improved systems and methods for processing read data from the sequenced oligos to recover the data encoded in the original oligo, including both address/index data and user data, are further described with regard to FIGS. 2-7.

In FIG. 1B, method 130 may be used to convert an analog read signal corresponding to a sequence of DNA bases back to the digital data unit that was the original target of the DNA storage process. In the example shown, the original digital data unit, such as a data file, was broken into data subunits corresponding to a payload size of the oligos, and the set of oligos corresponding to the subunits of the data unit may be reassembled into the original data unit. An example oligo format 140, including primers 142 and 148 that may be added to support the PCR amplification and sequencing, may include a payload 144 comprising a subunit of the data unit, a redundancy portion 146 for error correction code (ECC) data for that subunit, and an address portion 150 for determining the sequence of the payloads for reassembling the data block. In some configurations, Reed-Solomon error correction codes may be used to determine the redundancy portion 146 for payload 144.

At block 160, DNA base data signals may be read from the sequenced DNA. For example, the analog signal from the nanopore reader may be conditioned (equalized, filtered, etc.) and converted to a digital data signal for each oligo.

At block 162, multiple copies of the oligos may be determined. Through the amplification process, multiple copies of each oligo may be produced, and the decoding system may determine groups of the same oligo to process together.

At block 164, each group of the same oligo may be aligned, and consensus across the multiple copies may be determined. For example, a group of four copies may be aligned based on their primers, and each base position along the set of base values may have a consensus algorithm applied to determine a most likely version of the oligo for further processing, such as, where 3 out of 4 agree, that value is used.

At block 166, the primers may be detached. For example, primers 142 and 148 may be removed from the set of data corresponding to payload data 144, redundancy data 146, and address 150.

At block 168, error checking may be performed on the resulting data set. For example, ECC processing of payload 144 based on redundancy data 146 may allow errors in the resulting consensus data set for the oligo to be corrected. The number of correctable errors may depend on the ECC code used. ECC codes may have difficulty correcting errors created by insertions or deletions (resulting in shifts of all following base values). The size of the oligo payload 144 and portion allocated to redundancy data 146 may determine and limit the correctable errors and efficiency of the data format.

At block 170, the bases or base symbols may be inversely mapped back to the original bit data. For example, the symbol encoding scheme used to generate the DNA code may be reversed to determine corresponding sequences of bit data.

At block 172, a file or similar data unit may be reassembled from the bit data corresponding to the set of oligos. For example, address 150 from each oligo payload may be used to order the decoded bit data and reassemble the original file or other data unit.

FIG. 2 shows an improved DNA storage system 200 and, more specifically, an improved encoding system 210 and decoding system 240 for using multistage error correction using reference marks and soft information consensus to correct for insertion/deletion errors and/or mutation/erasure errors using multilevel encoding and decoding. In some configurations, encoding system 210 may be part of a first computer or storage system or device used for determining target binary data, such as a conventional binary data unit, and converting it to a DNA base sequence for synthesis into DNA for storage, and decoding system 240 may be part of a second computer or storage system or device used for receiving the data signal corresponding to the base sequence read from the DNA.

Encoding system 210 may include a processor 212, a memory 214, and a synthesis system interface 216. For example, encoding system 210 may be part of a computer or storage system or device configured to receive or access conventional computer data, such as data stored as binary files, blocks, data objects, databases, etc., and map that data to a sequence of DNA bases for synthesis into DNA storage units, such as a set of DNA oligos. Processor 212 may include any type of conventional processor or microprocessor that interprets and executes instructions. In some configurations, processor 212 may include a plurality of processors or processor cores configured to operate alone or in combination to execute one or more functions or sets of instructions described with regard to the other components of encoding system 210. Memory 214 may include a random-access memory (RAM) or another type of dynamic storage device that stores information and instructions for execution by processor 212 and/or a read only memory (ROM) or another type of static storage device that stores static information and instructions for use by processor 212. In some configurations, one or more components of encoding system 210 may be embodied in specialized logic and memory circuits configured for the functions described for encoding system 210 and may incorporate or operate in conjunction with processor 212 and memory 214. For example, one or more encoders, formatters, and/or insertion functions may be embodied in a specialized encoder circuit, such as a system on a chip (SOC), field programmable gate array (FPGA), application specific integrated circuit (ASIC), or similar circuit configuration, configured to operate alone or in conjunction with other encoder circuits and/or processor 212 and memory 214. Encoding system 210 may also include any number of input/output devices and/or interfaces. Input devices may include one or more conventional mechanisms that permit an operator to input information to encoding system 210, such as a keyboard, a mouse, a pen, voice recognition and/or biometric mechanisms, etc. Output devices may include one or more conventional mechanisms that output information to the operator, such as a display, a printer, a speaker, etc. Interfaces may include any transceiver-like mechanism that enables encoding system 210 to communicate with other devices and/or systems. For example, synthesis interface 216 may include a connection to an interface bus (e.g., peripheral component interconnect express (PCIe) bus) or network for communicating the DNA base sequences for storing the data to a DNA synthesis system. In some configurations, synthesis interface 216 may include a network connection using internet or similar communication protocols to send a conventional data file listing the DNA base sequences for synthesis, such as the desired sequence of bases for each oligo to be synthesized, to the DNA synthesis system. In some configurations, the DNA base sequence listing may be stored to conventional removable media, such as a universal serial bus (USB) drive or flash memory card, and transferred from encoding system 210 to the DNA synthesis system using the removable media.

In some configurations, a series of processing components 218 may be used to process the target binary data, such as a target data file, data block, or other data unit, into the DNA base sequence listing for output to the synthesis system. For example, processing components 218 may be embodied in encoder software and/or hardware encoder circuits. In some configurations, processing components 218 may be embodied in one or more software modules stored in memory 214 for execution by processor 212. Note that the series of processing components 218 are examples, and different configurations and ordering of components may be possible without materially changing the operation of processing components 218. For example, in an alternate configuration, additional data processing, such as a data randomizer to whiten the input data sequence, may be used to preprocess the data before encoding. In another configuration, user data from a target data unit may be divided across a set of oligos according to oligo payload size or other data formatting prior to applying any encoding or synchronization (or sync) marks may be added after ECC encoding. Other variations are possible.

In some configurations, processing the target data may begin with a run length limited (RLL) encoder 220. RLL encoder 220 may modulate the length of stretches in the input data. RLL encoder 220 may employ a line coding technique that processes arbitrary data with bandwidth limits. Specifically, RLL encoder 220 may bound the length of stretches of repeated bits or specific repeating bit patterns so that the stretches are not too long or too short. By modulating the data, RLL encoder 220 can reduce problematic data sequences that could create additional errors in subsequent encoding and/or DNA synthesis or sequencing. In some configurations, RLL encoder 220 or a similar data modulation component may be configured to modulate the input data to ensure that data patterns used for syntax references do not appear elsewhere in the user data encoded in the oligo.

In some configurations, symbol encoder 222 may include logic for converting binary data into symbols based on the four DNA bases (adenine (A), cytosine (C), guanine (G), and thymine (T)). In some configurations, symbol encoder 222 may encode each bit as a single base pair, such as 1 mapping to A or T and 0 mapping to G or C. In some configurations, symbol encoder 222 may encode two-bit symbols into single bases, such as 11 mapping to A, 00 mapping to T, 01 mapping to G, and 10 mapping to C. More complex symbol mapping can be achieved based on multi-base symbols mapping to correspondingly longer sequences of bit data. For example, a two-base symbol may correspond to 16 states for mapping four-bit symbols, or a four-base symbol may map the 256 states of byte symbols. Multi-base pair symbols could be preferable from an oligo synthesis point of view. For example, synthesis could be done not on base pairs but on larger blocks, like ‘bytes’ correlating to a symbol size, which are prepared and cleaned up earlier (e.g., pre-synthesized) in the synthesis process. This may reduce the amount of synthesis errors. From an encoder/decoder point of view, these physically larger blocks could be treated as symbols or a set of smaller symbols.

In some configurations, data encoder 224 may include logic for encoding the user data unit using one or more error correction schemes and may encode user data units across multiple oligos. For example, encoding system 210 may use low-density parity check (LDPC) codes constructed for the oligo size and/or larger codewords than can be written to a single oligo. In some configurations, data across multiple oligos may be aggregated to form the desired codewords. Similarly, parity or similar redundancy data may not need to be written to each oligo and may instead be written to only a portion of the oligos or written to separate parity oligos that are added to the oligo set for the target data unit. In some configurations, ECC encoding may then be nested for increasingly aggregated sets of oligos, where each level of the nested ECC corresponds to increasingly larger codewords comprised of more oligos. Encoding system 210 may include one or more oligo aggregators and corresponding iterative encoders. For example, each oligo may be encoded with its own redundancy data for a first level iterative decoder and/or rely solely on reference mark insertion/deletion correction and consensus across oligo copies for primary oligo data decoding. Then one or more aggregate levels of encoding/decoding may be applied to groups of oligos. For example, single level ECC encoding may use first level oligo aggregator and first level iterative encoder for codewords of 200-400 oligos. A two-level encoding scheme would use first and second level oligo aggregators for and corresponding first and second level iterative encoders, such as for 200 oligo codewords at the first level and 4000 oligo codewords at the second level.

Data encoder 224 may append one or more parity bits or similar redundancy data to the sets of codeword data for later detection whether certain errors occur during data reading process. For instance, an additional binary bit (a parity bit) may be added to a string of binary bits that are moved together to ensure that the total number of “1” in the string is even or odd. The parity bits may thus exist in two different types: an even parity in which a parity bit value is set to make the total number of “1” in the string of bits (including the parity bit) to be an even number, and an odd parity in which a parity bit is set to make the total number of “1” in the string of bits (including the parity bit) to be an odd number. In some examples, data encoder 224 may implement a linear error correcting code, such as LDPC codes or other turbo codes, to generate codewords that may be written to and more reliably recovered from the DNA medium. In some configurations, resulting parity or similar redundancy data may be stored in parity oligos designated to receive the redundancy data for the set of oligos that make up the codeword data. This additional parity data may be encoded using RLL encoder 220, symbol encoder 222, oligo formatter 226, and/or reference mark logic.

In some configurations, oligo formatter 226 may include logic for allocating portions of the target data unit to a set of oligos. For example, oligo formatter 226 may be configured for a predetermined payload size for each oligo and select a series of symbols corresponding to the payload size for each oligo in the set. In some configurations, the payload size may be determined based on an oligo size used by the synthesis system and any portions of the total length of the oligo that are allocated to redundancy data, address data, reference mark data, or other data formatting constraints. For example, a 150 base pair oligo using two-base symbols may include an eight-base addressing scheme and six four-base sync marks, resulting in 118 base pairs of the target data allocated to each oligo. In some configurations, oligo formatter 226 may insert a unique oligo address or oligo index (including the oligo address) for each oligo in the set, such as at the beginning or end of the data payload. The oligo address may allow the encoding and decoding systems to identify the data unit and relative position of the symbols in a particular oligo relative to the other oligos that contribute data to that data unit. For example, decoding system 240 may use position information corresponding to the oligo addresses to reassemble the data unit from a set of oligos in an oligo pool containing one or more data units. In some configurations, this position information may be used to order and assemble aggregate blocks for one or more levels of ECC encoding and decoding. In some configurations, rather than including the oligo index in a data sequence separate from the user data payload, such as a header or footer of the oligo data, the oligo index may be embedded or encoded in the reference marks distributed through the data payload. This may allow the reference marks to double as oligo index space, reducing overall data overhead, and allow for redundant copies of the oligo index to be more reliably encoded in the oligo.

In some configurations, oligo index formatter 228 may include logic to assemble the oligo index data to be included in the oligo. In prior oligo data formats, oligo index formatter 228 may operate as part of oligo formatter 226 to collect and format the oligo index data for the sequence of base pairs that would store the index, such as a specific set of base pairs within the oligo header. In configurations that embed the oligo index in a distributed fashion, such as embedded within the sequence of reference marks, oligo index formatter 228 may operate separately from oligo formatter 226 to conform with the reference mark format determined by reference mark encoder 230, reference mark formatter 232, and reference mark inserter 234. Oligo index formatter 228 may determine a sequence or set of base pairs that correspond to the oligo index data, such as the oligo address, one or more other syntactical parameters, and/or encoding specific to the oligo index (e.g., oligo index cyclic redundancy check (CRC) or parity bits). In some configurations, the oligo index data may include only the oligo address, with or without redundancy data. The set of data for the oligo index may include an oligo address value of a predetermined bit length to provide unique addresses to the set of oligos in a DNA oligo pool. In some configurations, the oligo index value may be mapped or translated into a set of reference patterns for encoding in the reference marks of the oligo. For example, one or more reference patterns may be mapped to oligo address values, with or without additional redundancy data, such that the selected reference pattern encodes the oligo address.

In some configurations, reference mark encoder 230 may include logic for determining a pattern of values to be used for reference marks. For example, a series of base pairs placed at rigid, predetermined intervals or frequency may conform to a known sequence for detecting the presence of the reference marks interspersed with user data over the length of the oligo. In some configurations, each reference mark may be a single base pair and, thus, would not be inherently distinguishable from user data encoded using that base pair. However, when aggregated across a series or set of reference marks, the reference marks may be identified as syntactic reference pattern separate from the user data they provide a reference for. By using a rigid interval or frequency of a fixed number of user data base pairs between reference mark base pairs, a pattern similar to the timing structure used in the reading and writing of moving media, such as rotating magnetic/optical disks or linear magnetic tapes, may be established. In some configurations, similar encoding and logics from timing marks in moving media may be used for reference marks. For example, a convolutional code, including hash encoded decoded by greedy exhaustive search (HEDGES) ECC, that is configured for detection and recovery across a series of reference marks, may be selected. In some configurations, reference mark encoder 230 may include a cyclic redundancy check (CRC) code that may be used to verify the sequence in the reference marks during the decoding process. In some configurations, reference mark encoder 230 may select or assemble reference patterns from a reference set 238 that includes a fixed set of known or predetermined reference patterns or reference pattern segments (e.g., each reference pattern or reference pattern segment corresponding to a unique convolutional code instance) that provide detectable patterns for reestablishing the data frequency during decoding without unduly increasing the error rates in DNA synthesis.

In some configurations, reference mark encoder 230 may operate on the oligo index data set received from oligo index formatter 228. For example, the oligo index data may be modulated and encoded using a convolutional code to be reliable distinguished from the user data as reference marks during decoding. That is, the oligo index data may form the basis of the reference mark pattern, and reference mark encoder 230 may be configured to receive and encode the oligo index data set for generation of the reference marks. In some configurations, the oligo index data may be mapped, with or without redundancy data, to reference pattern segments selected from reference set 238. Reference mark encoder 230 may provide the oligo address encoded as a series of reference pattern segments to reference mark formatter 232 for determining the configuration of the multiple reference pattern segments to be stored across the reference marks.

In some configurations, reference mark formatter 232 may include logic for formatting reference marks for insertion at predetermined intervals among the data symbols. For example, reference marks may be inserted every X base pairs to divide the data in the oligo into a predetermined number of shorter data segments with a timing frequency of 1/X and a resulting code rate of 1/(X+(base pairs in each reference mark)). In some configurations, each reference mark may comprise a single base pair as described for reference mark encoder 230 for a code rate of 1/(X+1). In some configurations, a code rate of 0.5(1/(1+1), alternating a base pair of user data with a base pair reference mark) may be selected to provide a desired likelihood of reference recovery and a high likelihood of identifying and correcting insertion and deletion errors to return the user data to a desired timing/data pattern with only mutation and/or erasure errors to be addressed through user data ECC. In an example configuration, an oligo may have a payload space of 140 base pairs, and formatting the reference marks at a 0.5 code rate would result in 70 base pairs of user data alternating with 70 base pairs of reference marks. The predetermined sequence and frequency of the reference marks may be used during the decoding process to determine and evaluate user data segments within an oligo to better detect and localize insertions and deletions that are difficult for error correction codes to detect or correct. For example, decoding system 240 may detect reference marks and correct symbol alignment prior to attempting iterative decoding with ECC. Use of reference marks is further described below with regard to decoding system 240.

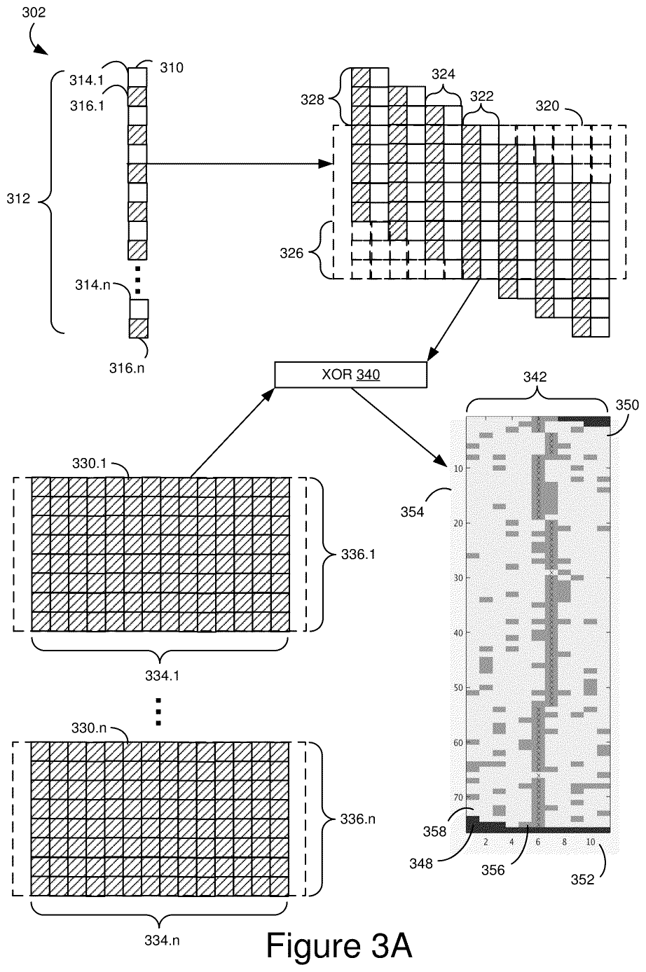

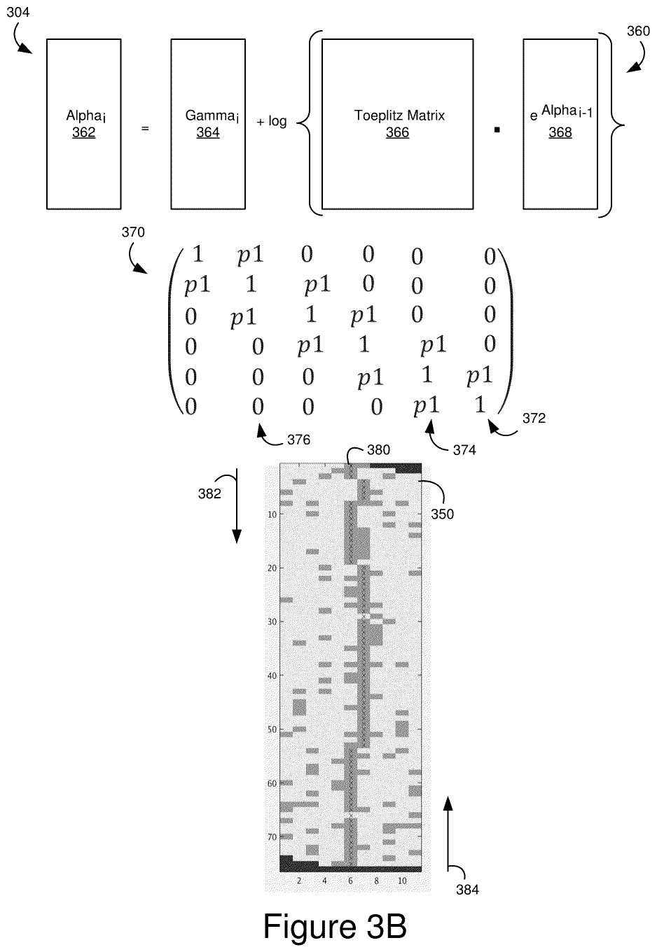

Reference mark formatter 232 may be configured to determine the number of reference pattern segments and configuration of those segments in the encoded reference pattern. For example, where the reference pattern is based on assembling a plurality of reference pattern segments, reference mark formatter 232 may determine the number of copies and the positions and/or translations of those segments across the reference marks. As shown in FIG. 3A, an example set of oligo data 310 may include user data base pairs 314.1-314.n alternating with reference mark base pairs 316.1-316.n. In the example shown, the oligo index comprises a base pair sequence 312 with a predetermined length for encoding user data alternating with reference marks. The sequence of reference marks 316.1-316.n may include a single reference pattern or a sequence of reference pattern segments assembled into a larger reference pattern. In some configurations, one or more reference pattern segments may be used to encode the oligo index or oligo address.

In some configurations, reference mark inserter 234 may include logic to insert the sequence of reference marks into the user data for determining the DNA sequence to be synthesized. For example, reference mark inserter 234 may operate according to the frequency configured in reference mark formatter 232 to insert the sequence of base pairs determined by reference mark encoder 230 and ordered copies from reference mark formatter 232 between corresponding portions of the user data. In the example using a code rate of 0.5, reference mark inserter 234 may alternate selecting a next base pair from the encoded user data with a next base pair from the reference marks to interleave single base pairs of user data with single base pair reference marks. In other configurations, a single base pair reference mark may be inserted after a plurality of user data base pairs, such as a larger (multi-base pair) symbol or segment size.

The resulting DNA base pair sequence corresponding to the encoded target data unit may be output from processing components 218 as DNA data 236. For example, the base pair sequences for each oligo in the set of oligos corresponding to the target data unit may be stored as sequence listings for transfer to the synthesis system. In some configurations, the base pair sequences may include the encoded data unit data formatted for each oligo, including address, sync mark, and redundancy data added to the user data for the data unit. The set of oligos may include a plurality of first level codeword sets and their corresponding parity oligos and, in some configurations, nested groups of first level codeword sets, second level codeword sets, and so on for as many levels as the particular recovery configuration supports.

Decoding system 240 may include a processor 242, a memory 244, and a sequencing interface 246. For example, decoding system 240 may be part of a computer or storage system or device configured to receive or access analog and/or digital signal read data from reading sequenced DNA, such as the data signals associated with a set of oligos that have been amplified, sequenced, and read from stored DNA media. Processor 242 may include any type of conventional processor or microprocessor that interprets and executes instructions. In some configurations, processor 242 may include a plurality of processors or processor cores configured to operate alone or in combination to execute one or more functions or sets of instructions described with regard to the other components of decoding system 240. Memory 244 may include a random-access memory (RAM) or another type of dynamic storage device that stores information and instructions for execution by processor 242 and/or a read only memory (ROM) or another type of static storage device that stores static information and instructions for use by processor 242. In some configurations, one or more components of decoding system 240 may be embodied in specialized logic and memory circuits configured for the functions described for decoding system 240 and may incorporate or operate in conjunction with processor 242 and memory 244. For example, one or more encoders, formatters, and/or insertion functions may be embodied in a specialized decoder circuit, such as a system on a chip (SOC), field programmable gate array (FPGA), application specific integrated circuit (ASIC), or similar circuit configuration, configured to operate alone or in combination with other decoder circuits and/or processor 242 and memory 244. Decoding system 240 may also include any number of input/output devices and/or interfaces. Input devices may include one or more conventional mechanisms that permit an operator to input information to decoding system 240, such as a keyboard, a mouse, a pen, voice recognition and/or biometric mechanisms, etc. Output devices may include one or more conventional mechanisms that output information to the operator, such as a display, a printer, a speaker, etc.

Interfaces may include any transceiver-like mechanism that enables decoding system 240 to communicate with other devices and/or systems. For example, sequencing interface 246 may include a connection to an interface bus (e.g., peripheral component interconnect express (PCIe) bus) or network for receiving analog or digital representations of the DNA sequences from a DNA sequencing system. In some configurations, sequencing interface 246 may include a network connection using internet or similar communication protocols to receive a conventional data file listing the DNA base sequences and/or corresponding digital sample values generated by analog-to-digital sampling from the sequencing read signal of the DNA sequencing system. In some configurations, sequencing interface 246 may include a messaging or application protocol interface for communicating with the DNA sequencing system that may be configured to automatically amplify and sequence samples from an oligo pool for generating read data. Decoding system 240 may be configured to both receive read data from the DNA sequencing system and initiate DNA processing by the sequencing system, which may include passing parameters to the DNA sequencing system. For example, amplification and sequencing may be executed iteratively based on target numbers of copies of oligos from the oligo pool and resulting decoding attempts and decoding system 240 may provide oligo counts and/or target parameters for the target number of oligo copies for one or more iterations. In some configurations, the DNA base sequence listing may be stored to conventional removable media, such as a universal serial bus (USB) drive or flash memory card, and transferred to decoding system 240 from the DNA sequencing system using the removable media.

In some configurations, a series of processing components 248 may be used to process the read data, such as a read data file or read data signal from a DNA sequencing system, to output a conventional binary data unit, such as a computer file, data block, or data object. For example, processing components 248 may be embodied in decoder software and/or hardware decoder circuits. In some configurations, processing components 248 may be embodied in one or more software modules stored in memory 244 for execution by processor 242. Note that the series of processing components 248 are examples and different configurations and ordering of components may be possible without materially changing the operation of processing components 248. For example, in an alternate configuration, additional data processing for reversing modulation or other processing from encoding system 216 and/or reassembly of decoded oligo data into larger user data units may be included. Other variations are possible.

Decoding system 240 may use a first stage of error correction targeting the elimination of insertion and deletion errors (which create shifts in all following base pairs in a sequence), followed by ECC error correction to address mutation or erasure errors. In some configurations, consensus correction may be used in the first stage to assist with reference mark timing and insertion/deletion correction and, more specifically, to use consensus correction for replacing base pairs lost in deletion errors (rather than just marking them as erasures). DNA media and sequencing face three main types of errors: deletion, insertion, and mutation. Mutation errors are most similar to the traditional errors in data storage and may efficiently be handled using ECC correction. Insertion and deletion errors affect the position of all following bits or symbols and may not be effectively handled by ECC. Therefore, preprocessing the oligo sequences for sequence position shifts and, where possible, correcting those position shifts may contribute to more efficient and reliable data reading. The preprocessing stage may reduce the error rate in the oligo sequences significantly prior to applying ECC correction, enabling more efficient ECC codes and more reliable retrieval of data using a first level of ECC encoding in nested ECC configurations. In some configurations, the preprocessing stage may include reference mark decoder 250, insertion/deletion correction 252, erasure identifier 254, oligo index decoder 256, oligo set sorter 258, and data consensus correction 260.

In some configurations, reference mark decoder 250 may include logic for comparing read data for an oligo to a plurality of different reference matrices to determine the most likely reference pattern for detecting and correcting insertions and deletions based on reference marks with a known frequency. For example, the read data may be processed through multiple attempts to determine a most likely path through correlation matrices corresponding to different possible reference patterns from reference set 238, and resulting scoring from those attempts may be used to select the most likely reference pattern to use for reference mark decoding and timing correction. In some configurations, reference mark decoder 250 may be configured to operate on a single copy of oligo read data and reliably determine the set of reference marks using a correlation matrix to align the possible reference marks and multiple copies of each reference pattern to determine the reference pattern that maximizes the quality of the timing information. As shown in FIG. 3A, a series of operations 302 may generate and populate a correlation matrix based on oligo read data 310. In some configurations, read data 310 may be used to generate a convolutional matrix 320. Convolutional matrix 320 may be based on filtering and stacking reference mark positions 316.1-316.n and user data positions 314.1-314.n from the read data to make alternating columns of the respective base pairs. In the example shown, one base pair of reference marks alternates with one base pair of user data, so filtering may be based on selecting the odd positions as user data values and the even positions as reference mark values. Each column includes a number of base pairs representing the user data portion or the reference mark portion of the oligo (e.g., half the length of the oligo for each type). The center pair of columns 322 comprise the full set of reference mark base pairs and user data base pairs. Each pair of columns in either direction is then offset by one row or base pair of that type (though this represents a two base pair offset when both reference marks and user data are considered). For example, columns 324 are offset by one row from columns 322. Where no read values are available due to the offset of the read data, placeholders 326 may be added. These are null values that are not valid portions of convolutional matrix 320 for subsequent calculations. Similarly, read data values 328 that fall outside of convolutional matrix 320 are not represented in the matrix and are shown merely for illustrating the data shift. The resulting convolutional matrix includes rows corresponding to pairs of positions along the oligo, and the columns correspond to single base pair offsets that, if no insertion or deletion errors were present, would alternate between reference mark base pairs and user data base pairs.

In some configurations, reference set 238 may include a set of unique and predetermined or fixed reference patterns used to encode the reference marks for each oligo. At this point in the processing, which reference pattern was used for the particular oligo may be unknown, and reference mark decoder 250 may sequentially attempt each reference pattern to determine which of the reference patterns was used to encode the reference marks. For example, reference set 238 may include 16 or more different reference patterns. In some configurations, reference set 238 may include reference pattern segments that may be processed in combinations and/or repeated in a plurality of positions to form the larger reference pattern. For example, 256 reference pattern segments may support different positions in the combined reference pattern that encodes an oligo index in addition to providing reference marks for error correction. Reference mark decoder 250 may use a series of reference matrices 330.1-330.n each corresponding to the different reference patterns in reference set 238. Unlike convolutional matrix 320, reference matrices 330 are constructed without offset. Each column 334.1-334.n repeats the reference pattern set of base pairs in reference matrix 330.1-330.n. Each row 336.1-336.n includes the same base pair values corresponding to that position in the reference pattern.

A series of exclusive-or (XOR) operations 340 may then be used to compare each base pair in convolutional matrix 320 with the corresponding matrix position in reference matrices 330.1-330.n. The results of these comparisons may populate a correlation matrix 350 with binary logical matrix values 342. The x-axis 352 denotes the columns of the correlation matrix. The central reference mark column from convolutional matrix 320 corresponds to 0 offset, and each column to the left or right corresponds to a one base pair offset in that direction. In the example shown, a band of 5 offset positions in each direction is being used, with 6 being the nominal (encoded) position of the reference marks, 1 being a 5 base pair shift generally corresponding to the direction of deletions, and 11 being a 5 base pair shift generally corresponding to the direction of insertions. The y-axis 354 denotes the rows of the correlation matrix and corresponds to reference mark positions along the oligo. Medium gray positions, such as block 356, correspond to a 0 value or base pair matches from the XOR comparison. Light gray positions, such as block 358, correspond to a 1 value or base pair mismatches from the XOR comparison. Dark gray positions in the corners of the matrix, such as block 348, correspond to the placeholder or null values that are outside the valid positions of the matrix (created by the data shifts in convolutional matrix 320). Correlation matrix 350 may be used to determine the most likely positions of insertions and deletions based on shifts in the matching values by one or more columns in each direction, as well as determining which of the reference patterns provides the best results and most likely reference pattern to use. Note that while convolutional matrix 320 and reference matrices 330.1-330.n are graphically illustrated with a small number of oligo positions, example correlation matrix 350 is based on a more common oligo size of 150 base pairs, yielding 75 base pair positions corresponding to reference marks at a 0.5 code rate.

As shown in FIG. 3B, a sequence of operations 304 may use a Viterbi-like algorithm 360 to analyze correlation matrix 350 and identify the most likely path through the sequence of reference positions. The algorithm applies one or more traverses of the rows of correlation matrix 350 to determine whether the reference position has shifted due to an insertion or deletion. In some configurations, algorithm 360 is a recursive Viterbi algorithm that uses the probability determined for a prior value in the matrix to determine the probability of the next value in the matrix in the direction of the traversal. Alphaᵢ values 362 may include a set of values corresponding to the next row in correlation matrix 350 to be analyzed. Gammaᵢ values 364 may correspond to a set of values for the column shift or offset of the next row. The offset values may be modified by a probabilistic term based on the log of the exponential function of the prior Alpha values 368 (e^{Alpha}ᵢ₋₁) multiplied by a Toeplitz matrix 366. Toeplitz matrix 366 represents the probabilities of moving from one column to the other (insertions or deletions shifting reference mark alignment) and limits the probable movements to a desired range. An example Toeplitz matrix 370 comprises a central diagonal 372 corresponding to no shift and represented by constant values of 1. The p1 values 374 correspond to a one position shift in either direction. The p1 value may be selected to represent a likelihood of position shift. For example, a p1 value may be 0.03 for one position shift in each direction.

The output of Viterbi algorithm 360 may be a set of probabilities for each row, with the highest probability value indicating the most likely shift or offset for that position (or no shift/offset if the highest probability corresponds to the central reference mark column). In the example shown, indicator (x) 380 in each row corresponds to the column selected for that row as the most likely shift value based on the highest probability value for that row. In some configurations, Viterbi algorithm 360 may be evaluated in multiple directions and summed or averaged to determine the most likely values for each reference position (row). For example, a first direction 382 may correspond to traversing correlation matrix 350 from the top row (first reference mark position in the oligo) to the bottom row (last reference mark position in the oligo). A second direction 384 may correspond to traversing correlation matrix 350 in the reverse or opposite direction from the bottom row (last reference mark position in the oligo) to the top row (first reference mark position in the oligo). The results from the two traversals may then be summed or otherwise evaluated to determine the most likely shift for each reference mark position. The resulting summation (or a single traversal if that is all that is used) results in probability values for each entry in the correlation matrix that may be referred to as soft information from the Viterbi algorithm traversal of the correlation matrix.

In some configurations, a compare function of reference mark decoder 250 may execute Viterbi algorithm 360 for each reference pattern in reference set 238 and corresponding reference matrices 330.1-330.n and calculate scores for each reference pattern. For example, the sum of columns may provide a relative likelihood value for that reference pattern. These Viterbi scores for each reference pattern may be compared, and the maximum value may indicate the most likely reference pattern. In some configurations, Viterbi scores may be calculated for each reference pattern segment, and corresponding sets of positions in the larger reference pattern and scores compared for each set of positions to determine the most likely reference pattern segment for that position. The resulting most likely reference pattern may be based on the ordered set of reference pattern segments that maximized their respective segment positions. In some configurations, Viterbi normalization may be modified for the comparison of the different reference patterns. For example, the Viterbi scores may not be normalized for each column to improve the comparison of different reference matrices. In some configurations, the Viterbi algorithm 360 may be executed iteratively in response to adjustments made to correct insertions and deletions until reference timing may be restored or the reference timing is determined to be unrecoverable. In some configurations, the Viterbi scores for the different reference patterns may be compared to one another, as well as to a threshold value for acceptable decoding. Based on the most likely reference pattern selected, the output of that correlation matrix and Viterbi analysis may be used for subsequent detection and correction of erasure errors to reestablish the timing of the selected reference pattern.

In some configurations, insertion/deletion correction 252 includes logic for selectively correcting the insertion and deletion errors in an oligo, where possible. For example, insertion/deletion correction 252 may use the output from reference mark decoder 250 to determine correctable insertion/deletion errors. For example, where an insertion or deletion error has occurred between sync marks, the position of subsequent segments may be corrected for the preceding shift in base pair positions to align the symbols in segments without insertions/deletions with their expected positions in the oligo. In some configurations, reference mark decoder 250 may enable insertion/deletion correction 252 to specifically identify likely locations of insertion and/or deletion errors at the base pair level due to the 0.5 code rate. In some configurations, insertion deletion correction 252 may be used following data consensus correction 260 where oligo copies are known. This may be the result of a first pass through oligo index decoder 256 and oligo set sorter 258 based on single copy data and/or where copies are sorted based on data that is not encoded in the reference marks.

In some configurations, erasure identifier 254 may flag segments of base pairs in the oligo as erasures in need of ECC error correction. For example, reference mark decoder 250 and related analysis may determine one or more base pairs between sync marks to be identified as erasures for error correction and/or data consensus correction 260. In some configurations, signal quality, statistical uncertainty, and/or specific thresholds for consensus may cause erasure identifier 254 to identify one or more segments as erasures because the processing by reference mark decoder 250 and/or data consensus correction 260 is inconclusive. For example, erasure identifier 254 may be configured to output an oligo that has had as many base pairs or symbols as possible positively identified as not containing insertion or deletion errors and identify (and localize) as erasures any base pairs that cannot be determined to be free of insertion or deletion errors prior to performing ECC error correction. Most commonly, placeholders inserted for identified deletions to restore reference mark timing may be identified as erasures. Note that mutation errors in DNA storage may be equivalent to the erasure errors ECC are configured to correct and need not be identified in the preprocessing stage. In some configurations, the insertion/deletion corrected output oligo base pair sequence and related erasure tags and/or soft information from reference mark decoder 250 may be the output of the preprocessing stage of decoding system 240.

In some configurations, the reference marks may also be configured to encode the oligo index in the reference pattern. For example, the reference marks may include a plurality of reference pattern segments that encode the oligo index value for the oligo index. In these configurations, the most likely reference pattern may be passed to oligo index decoder 256. Oligo index decoder 256 may include logic to determine the oligo index from the reference marks distributed through the oligo read data. For example, the read data for each oligo may include reference marks interspersed with the user data, and the oligo index data may be encoded in one or more sequences of the reference marks. In some configurations, the oligo index may be encoded using correlation to one or more reference patterns in the reference marks. For example, reference set 238 may include reference pattern segments that map to bit values for encoding the oligo address. In some configurations, the oligo address is segmented into multiple oligo address portions that are each encoded using different reference patterns. The series of reference pattern segments from the most likely reference pattern may be used to determine the corresponding oligo address segments. Those oligo address segments may then be reassembled into the oligo address. In some configurations, the oligo address and/or oligo address segments may include redundancy data. Oligo index decoder 256 may perform a redundancy check to determine whether the integrity of each oligo address segment and/or the oligo address as a whole is intact. If the redundancy check fails, operation may return to reference mark decoder, the oligo may be determined to be unreadable, or other error correction processing may be initiated. In some configurations, a CRC check may also be used for oligo index data verification after the oligo index segments are assembled. Oligo index decoder 256 may be configured to only return oligo index data with a high-level of confidence for use in further processing of the oligo read data.

In some configurations, oligo set sorter 258 may include logic to sort a received group of oligo data sequences into sets of copies. For example, the DNA amplification process may result in multiple copies of some or all oligos, and oligo set sorter 258 may sort the oligo data sequences into like sequences. Sorting may be based on tagging during the sequencing process, oligo address data (such as oligo address data from the oligo index decoder 256), and/or statistical analysis of sequences (or samples thereof) to determine repeat copies of each oligo. Note that different copies may include different errors and, at this stage, exact matching of all bases in the sequence may not be the sorting criteria. In this regard, each input oligo may generate a set of one or more oligo copies that correspond to the original input oligo data but may not be identical copies of that data or of one another, depending on when and how the errors were introduced (thus the need for error correction). Oligo set sorting to determine the number of multiple copies may be performed prior to stage one error correction and independent of the ability to successfully decode the oligo address if there are other indicators with sufficient reliability to sort the oligos. A set of oligo copies may be processed together, particularly through the first stage of processing, to determine or generate a best copy of the oligo for ECC processing. In some configurations, the best copy of the oligo may also be passed with the soft information aggregated from the multiple copies for use in ECC processing. Oligo set sorter 258 may also determine the number of copies of each oligo in a set of read data. In some configurations, read data sets for the same oligo pool may be received iteratively based on multiple amplification and/or sequencing operations and oligo set sorter 258 may identify and manage the oligo copies and corresponding copy counts as additional read data is received.

In some configurations, data consensus correction logic 260 may include one or more logic functions for using comparison of multiple preprocessed copies and may include copies that have had their reference mark timing recovered to reduce the number of erasure errors and/or resolve inconclusive reference mark corrections. In some configurations, data consensus correction logic 260 may use consensus averaging based on soft information from reference mark decoder 250 rather than direct comparison of base pair values from the most likely paths of each copy. For example, correlation analysis across more than two copies of an oligo may allow statistical methods and soft information values to be compared to a correction threshold for deleting inserted base pairs, inserting padding or placeholder base pairs (which may be identified as erasures by erasure identifier 254), and/or correcting mutation errors that appear in a minority of copies. The correction threshold may depend on the number of copies being cross-correlated, decoder signal-to-noise ratio (SNR), size of the insertion/deletion event, a reliability value of the statistical method, and/or the error correction capabilities of the subsequent ECC processing, including the any nested ECC. In some configurations, data consensus correction logic 260 may be included as part of the preprocessing stage of decoding system 240.

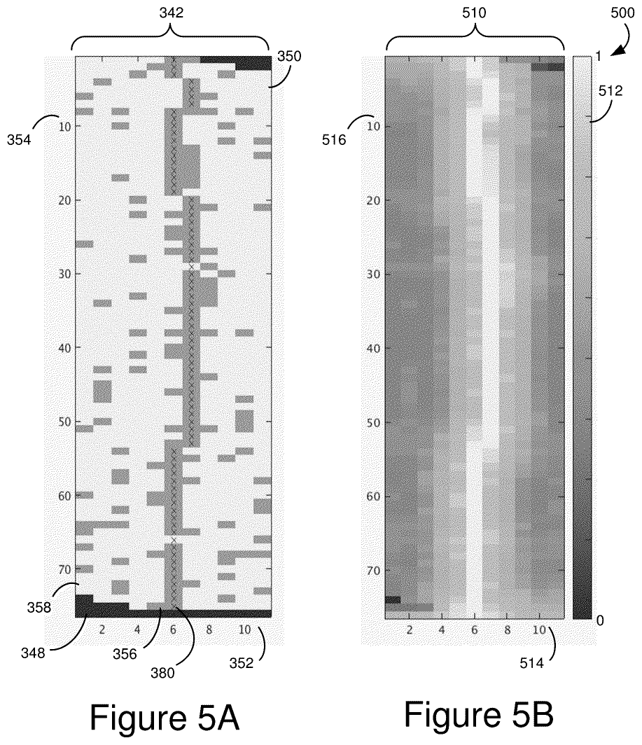

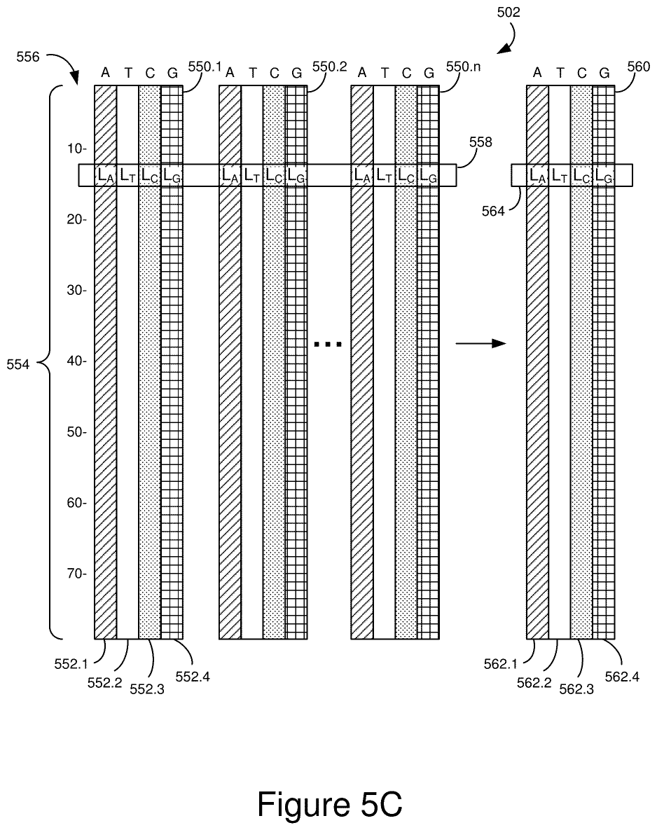

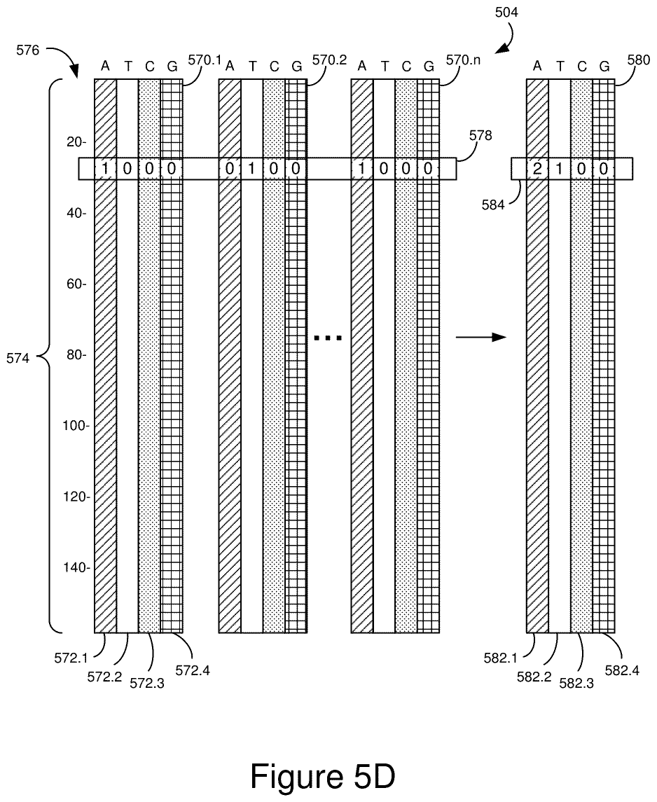

As shown in FIGS. 5A, 5B, and 5C, soft information from Viterbi algorithm processing of the correlation matrix from reference mark decoder 250 may be used for soft information consensus correction. For example, in FIG. 5A, correlation matrix 350 may be processed for each copy of the oligo with a resulting matrix of probability value entries that correlate to a most likely path 380 for that oligo copy. Correlation matrix 350 may correspond to the likelihood of position shifts based on the corresponding probability values being closer to 0 (medium gray entries 356) or 1 (light gray entries 358). However, the underlying probability values or soft information represent a larger spectrum of values. As shown in FIG. 5B, a soft value map 500 of correlation matrix 350 shows a wider range of gray-scale coded probability values 510. Soft information value map 500 may correspond to the matrix of probability values underlying correlation matrix 350. Scale 512 provides a gray-scale spectrum of probability values from 0 to 1 where the lightest gray value corresponds to a maximum likelihood value and the darkest gray value corresponds to a lowest likelihood value. Soft value map 500 is a way of visualizing the set of soft information from correlation matrix 350 and comprises the same matrix with position shifts on x-axis 514 and reference positions for the oligo on the y-axis. Note that the same type of correlation matrix and soft information map could be made for the full set of oligo positions, including both reference and oligo payload data.

FIG. 5C shows a set 502 of aggregate base pair probability values for multiple copies of the oligo. For example, the probability values from correlation matrix 350 may be mapped to a likelihood of each base pair for that position in the oligo. Each base pair matrix 550.1-550.n may include the probability of each base pair in each position aggregated across each row of correlation matrix 350. For example, each set of values 554 may include the likelihood of that base pair 552.1-552.4 at that position 556 (e.g., 1-80) along the length of the oligo. An example set of probability values (L_{A}, L_{T}, L_{C}, L_{G}) 558 for the likelihood at the same positions may be determined from correlation matrix 350 for each copy of the oligo. The sets of probability values for each base pair matrix 550.1-550.n may be aggregated into a consensus average matrix 560 that includes the aggregate value of each base pair 562.1-562.4 and positions 554 across the oligo copies. For example, the set of probability values 558 may then be aggregated into a consensus average set of values 564. The example base pair matrices are shown for reference mark positions, but similar base pair matrices could be constructed for all base pair positions in the oligo copies. In the example, shown, columns 552.1 and 562.1 correspond to the likelihood values for an A base pair at each oligo position, columns 552.2 and 562.2 correspond to the likelihood values for a T base pair at each oligo position, columns 552.3 and 562.3 correspond to the likelihood values for a C base pair at each oligo position, and columns 552.4 and 562.4 correspond to the likelihood values for a G base pair at each oligo position. Equivalent base pair matrices 550.1-550.n may be generated from corresponding correlation matrices for each copy of the oligo. These matrices and the soft information values they contain may be consensus averaged across the multiple copies to determine a most likely base pair value for each position along the length of the oligo as shown in consensus average matrix 560. This may be selectively determined for the reference mark positions only or for all base pair positions.

FIG. 5D shows a set 504 of aggregate base pair hard decision values for multiple copies of the oligo. For example, the maximum likelihood from among the probability values from correlation matrix 350 may be mapped to a most likely base pair for that position in the oligo. Each base pair matrix 570.1-570.n may include a 1 or a 0 for each node depending on whether that base pair is the most likely base pair for that position along the oligo. For example, each set of values 574 may include a 1 for the base pair among base pairs 572.1-572.4 that is most likely at that position 576 (e.g., 1-160) along the length of the oligo. An example set of hard decision values 578 for the same positions may be determined from correlation matrix 350 for each copy of the oligo. The sets of probability values for each base pair matrix 570.1-570.n may be aggregated into a consensus average matrix 580 that includes the aggregate value of each base pair 582.1-582.4 and positions 574 across the oligo copies. For example, the set of hard decision values 578 may be aggregated into a consensus set of values 584. In some configurations, consensus average matrix 580 may further include division of the aggregate values by the number of copies to determine consensus average values that are probabilistic or soft information. For example, consensus set of values 584 based on three copies of the oligo may be represented as soft information by 0.66, 0.33, 0.0, and 0.0. The example base pair matrices are shown for all positions (reference and oligo payload data), but similar base pair matrices could be constructed for reference positions only in the oligo copies. In the example, shown, columns 572.1 and 582.1 correspond to the likelihood values for an A base pair at each oligo position, columns 572.2 and 582.2 correspond to the likelihood values for a T base pair at each oligo position, columns 572.3 and 582.3 correspond to the likelihood values for a C base pair at each oligo position, and columns 572.4 and 582.4 correspond to the likelihood values for a G base pair at each oligo position. Equivalent base pair matrices 550.1-550.n may be generated from corresponding correlation matrices for each copy of the oligo. Using hard decision matrices to generate aggregate soft information may be most valuable when there are greater than two copies of the oligo and the value of the consensus average from base pair hard decisions may increase in accuracy relative to the Viterbi soft information aggregates as the number of copies of the oligo increases. These matrices and the soft information values they contain may be consensus averaged across the multiple copies to determine a most likely base pair value for each position along the length of the oligo as shown in consensus average matrix 580. This may be selectively determined for the reference mark positions only or for all base pair positions.

In some configurations, data consensus correction 260 may be configured with multiple consensus functions based on different aggregations of soft information. For example, a first consensus function may be based on reference soft information 260.1, where the soft information for the correlation of reference mark positions is used across the multiple copies to determine the most likely path and corresponding base pair values for the reference mark positions. A second consensus function may be based on base pair soft information 260.2, where the soft information for the correlation of base pair hard decisions in the oligo is used across the multiple copies to determine the most likely path and corresponding base pair values for all positions. In some configurations, these different consensus functions may be selectively used based on the number of available copies. For example, a threshold value may be applied to the number of copies in the read data set where one consensus function (such as the reference consensus function) is more reliable at lower copy numbers. Data consensus correction 260 may apply the one consensus function to determining and correcting the reference mark base pairs with copy numbers below the threshold and apply another consensus function for determining and correcting the payload data base pairs if the threshold is met (or exceeded). In some configurations, multiple consensus functions may be applied to base pair consensus decisions. For example, the aggregate soft information from each consensus function may be selectively combined for making the most likely base pair decision. In some configurations, a weighting factor may be used to balance the relative contributions of the different consensus functions. For example, a weighting factor may be a ratio value split between or among the consensus functions for determining the relative contributions of the aggregate soft information from the two or more functions for each position. In some configurations, the weighting factor may be variable based on the number of copies being used for the consensus determination. For example, at low numbers of copies, the reference mark consensus function may be weighted to contribute more to the likelihood decision and the ratio allocated to the full base pair or oligo payload consensus function may increase as the number of copies increases.

In some configurations, one or more iterative data decoders, including oligo iterative data decoder 262, may be configured to process the output from the preprocessing stage of decoding system 240. For example, a single “best guess” copy of each unique oligo in a set of oligos for a data unit, including erasure flags and/or soft information, may be passed from preprocessing to ECC decoding. In some configurations, reference marks, address fields, and other formatting data may be removed or ignored by decoding system 240 during ECC processing. Decoding system 240 may use one or more levels of ECC decoding based on aggregating the data from a number of oligos (unique oligos rather than copies of the same oligo). For example, decoding system 240 may use LDPC codes constructed for larger codewords than can be written to or read from a single oligo. Therefore, data across multiple oligos may be aggregated to form the desired codewords. Similarly, parity or similar redundancy data may not be retrieved from each oligo and may instead be read from only a portion of the oligos or from separate parity oligos in the oligo set for the target data unit. In some configurations, ECC decoding may then be nested for increasingly aggregated sets of oligos, where each level of the nested ECC corresponds to increasingly larger codewords comprised of more oligos. Decoding system 240 may include one or more oligo aggregators and corresponding iterative decoders. For example, single level ECC encoding may use first level oligo aggregator and first level iterative encoder for codewords of 200-400 oligos. A two-level encoding scheme would use first and second level oligo aggregators and corresponding first and second level iterative encoders, such as for 200 oligo codewords at the first level and 4000 oligo codewords at the second level.

In some configurations, iterative data decoders may help to ensure that the states at the codeword satisfy the parity constraint by conducting parity error checking to determine whether data has been erased or otherwise lost during data read/write processes. It may check the parity bits appended by data encoder 224 during the data encoding process, and compare them with the base pairs or symbols in the oligo sequences aggregated by the corresponding oligo aggregators. Based on the configuration of data encoder 224 in the data encoding process, each string of recovered bits may be checked to see if the “1” total to an even or odd number for the even parity or odd parity, respectively. A parity-based post processor may also be employed to correct a specified number of the most likely error events at the output of the Viterbi-like detectors by exploiting the parity information in the coming sequence. In some configurations, iterative data decoder 262 may use soft information received from preprocessing to assist in decode decision-making. When decode decision parameters are met, the codeword may be decoded into a set of decoded base pair and/or symbol values for output or further processing by symbol decoder 272, RLL decoder 274, and/or other data postprocessing.

In some configurations, oligo iterative decoder 262 may decode the oligo payload data for each oligo based on the aggregate soft information from multiple copies of each oligo. The decoded payload data from the set of copies may then be used to assemble a larger decode block, referred to as a hyperblock. Hyperblock assembly logic 264 may include logic to aggregate the “best copy” of the payload data for each oligo determined by oligo iterative decoder 262 or, in other configurations, directly from data consensus correction logic 260. Hyperblock assembly logic 264 may also receive oligo address information or other metadata for each oligo that identifies the position of that oligo in the sequence of oligos that make up the hyperblock. In some configurations, hyperblock assembly logic 264 may assemble the hyperblock by writing the sequence of oligo payloads into oligo data storage 266. For example, hyperblock assembly logic 264 may write the oligo payloads in order to a buffer memory of a data storage device, such as a hard disk drive or solid state drive including a non-volatile storage medium in which to store the data. In some configurations, the hyperblock may be sized to conform with a block size of the read channel of a data storage device and/or its memory buffer to enable the data storage device to more easily store and process the received data. Once the payload data from all oligos in the hyperblock are assembled, the hyperblock may be passed to or accessed by hyperblock decoder logic 270.

In some configurations, hyperblock decoder logic 270 may include one or more decoders and verification logic for determining whether the hyperblock can be successfully decoded based on the set of oligo payload data received from the oligo processing and decoding. For example, hyperblock decoder logic 270 may be configured to execute iterative decoding for a codeword sized for the aggregate block size and determine successful decoding based on ECC metrics and/or a CRC check. Hyperblock decoder logic 270 may include one or more iterative decoders 270.1 configured for ECC processing of one or more aggregate levels and corresponding codewords. For example, iterative decoder 270.1 may be configured as described above with regard to oligo iterative decoder 262 and iterative decoders, such as LDPC decoders, more generally. Hyperblock decoder logic 270 may include logic for verifying the successful decoding of the data in the hyperblock. For example, CRC 270.2 may provide a simple and reliable way to check if the decoded codeword is correct or it is a near codeword. CRC 270.2 may be implemented as a division of the codeword on a primitive polynomial in some Galois field. The CRC value may be determined for each binary data unit and added by the originating system or encoding system 210. For example, the remainder of the division may be stored in the codeword information for the later CRC check after decoding. CRC 270.2 may be particularly advantageous for DNA storage, where error rate is high and near codeword detection is more probable.

In some configurations, hyperblock logic 270 may include logic for iteratively cycling through additional amplification and sequencing in response to unsuccessful decoding, such as decode failures or CRC check failures. For example, cycle logic 270.3 may process the decoder metrics and/or CRC check output to determine whether the hyperblock has been successfully decoded. If it has, the decoded data may be passed to symbol decoder 272 for further processing or returned as a decoded data unit or portion of a data unit. If the error correction capability of iterative decoder 270.1 was insufficient to recover the codeword or CRC 270.2 indicates unsuccessful decoding, then cycle logic 270.3 may initiate additional processing by the DNA sequencing system to increase the number of copies of oligos from the data pool in an effort to improve the accuracy of the consensus algorithms and resulting best guess oligo payloads. In some configurations, cycle logic 270.3 may send a signal through sequencing interface 246 to initiate further amplification and/or sequencing to generate additional read data for the same oligo pool and corresponding set of oligos. For example, cycle logic 270.3 may send a request message for additional read data identifying the oligo pool or set of oligos. In some configurations, cycle logic 270.3 may include logic for determining a magnitude of the request for additional oligo copies. Each additional copy of an oligo may represent a signal gain from the perspective of iterative decoder 270.1 and an error rate of the hyperblock data may be used to determine an increment of the desired increase in signal. For example, cycle logic 270.3 may determine an error rate in the hyperblock data from the ECC metrics and may compare the error rate to the error correction capability of iterative decoder 270.1 to determine a target number of additional copies of the oligos to be generated. As noted elsewhere, oligo copies are generated randomly from the oligo pool and result in a distribution of numbers of copies across the oligos in the pool. Therefore, a target number of copies or increases in number of copies may be used to denote a total number of new oligo sequences to be generated or a target mean or median for the distribution of copies that may be used to determine the additional sequencing process and volumes. For example, cycle logic 270.3 may send a target number of new oligo sequences as a parameter value through sequencing interface 246 for initiating the additional sequencing. The additional read data may be received by decoding system 240, the new oligo copies processed, and their resulting soft information combined with the prior set of read data to update the hyperblock oligo payload values. This process may be done iteratively until the hyperblock is successfully decoded or a threshold is reached that indicates that additional copies are not sufficiently improving the decoding or a resource cap for amplification and sequencing has been reached.

In some configurations, symbol decoder 272 may be configured to convert the DNA base symbols used to encode the bit data back to their bit data representations. For example, symbol decoder 272 may reverse the symbols generated by symbol encoder 222. In some configurations, symbol decoder 272 may receive the error corrected sequences from hyperblock decoder logic 270 and output a digital bit stream or bit data representation. For example, symbol decoder may receive a corrected DNA sequence listing for one or more codewords corresponding to the originally stored data unit and process the corrected DNA sequence listing through the symbol-to-bit conversion to generate a bit data sequence. In some configurations, RLL decoder 274 may decode the run length limited codes encoded by RLL encoder 220 during the data encoding process. In some configurations, the data may go through additional post-processing or formatting to place the digital data in a conventional binary data format.

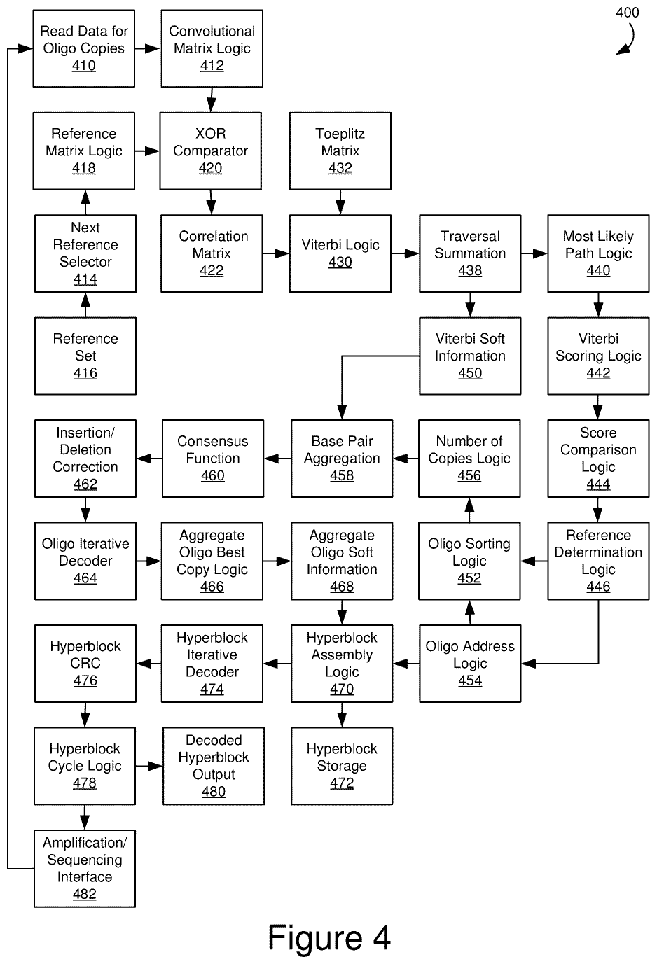

FIG. 4 includes an oligo data processing system 400 using soft information from multiple copies of an oligo for consensus correction and then aggregating and decoding a hyperblock to determine whether additional oligo copies are needed. In some configurations, blocks 410-446 may include logic and memory configured to execute the functions of reference mark decoder 250 in FIG. 2 and associated operations described with regard to FIGS. 3A and 3B. Block 454 may include logic and memory configured to execute functions of oligo index decoder 256 and block 452 may include logic and memory configured to execute functions of oligo set sorter 258. Blocks 456-462 may include logic and memory configured to execute functions of insertion/deletion correction 252, erasure identifier 254, and data consensus correction 260. Block 464 may include logic and memory configured to execute functions of oligo data decoder 262. Blocks 466-482 may include logic and memory configured to execute functions of hyperblock decoder logic 270. Blocks 410-482 may be embodied in hardware circuits and/or software modules executed using a processor and memory, such as processor 242 and memory 244 in FIG. 2.

At block 410, read data for an oligo copy may be received in memory. This read data may include an initial read of oligo sequences, as well as subsequent iterations of additional read data for the same oligo pool. Convolutional matrix logic 412 may generate a convolutional matrix based on separating reference mark positions and user data positions into alternating columns and then offsetting those pairs of columns by one base pair for a sequence of offset positions in both directions. Next reference selector 414 may select a target reference pattern from reference set 416 to use for a first iteration of scoring the most likely path through a corresponding correlation matrix for the convolutional matrix and reference matrix based on the selected reference pattern. Reference matrix logic 418 may access or generate a corresponding reference matrix for the selected matrix pattern, such as by repeating the reference pattern across the columns of the reference matrix. XOR comparator 420 may include logic to execute an XOR comparison of each corresponding value in the convolutional matrix and the two reference matrices to populate a correlation matrix 422. For example, XOR comparator 420 may execute XOR logic between the values from each row of the matrices and return a 0 value for matched base pairs and a 1 value for different base pairs. Example correlation matrix 350 is shown in FIG. 3A, where the light gray areas indicate different base pairs, and the medium gray areas indicate matched base pairs. At this stage, x-indicators 380 for the most likely path may not yet be determined.

Viterbi logic 430 may operate on correlation matrix 422 to determine probability values for each correlation value in the matrix based on traversing the matrix with a Viterbi algorithm. As described above, Viterbi logic 430 may be configured with a Toeplitz matrix 432 to bias the probabilities for single offset changes from the offset position of the prior reference mark position. Viterbi logic 430 may determine a highest probability among the values in each row of correlation matrix 422, as indicated by x-indicators 380 in example correlation matrix 350. In some configurations, Viterbi logic 430 may execute for multiple traverses of correlation matrix 422 and/or portions thereof based on different reference pattern segments encoded in the reference marks. In some configurations, Viterbi logic 430 may be executed through multiple traversals, such as a first (alpha) direction traversal and a second (beta) direction traversal. The resulting probability values for each matrix position may then be summed or otherwise combined by traversal summation 438. Each Viterbi operation may result in a set of probability data for correlation matrix 422 that may be output as Viterbi soft information 450. For example, Viterbi soft information 450 may include a matrix of probability values corresponding to the entries in correlation matrix 422. In some configurations, Viterbi logic 430 may operate on other configurations of correlation matrices, including correlations between copies of an oligo, where another copy of the oligo or a previously determined best copy from multiple copies is used to generate the reference matrix.

Most likely path logic 440 may use the probability values determined by Viterbi logic 430 directly or through the summation of multiple traversals of correlation matrix 422 to determine the most likely value (and corresponding offset column) for each row in correlation matrix 422. For example, the highest value in each row of example correlation matrices 350 may be annotated with indicator (x) 380 for the highest values, as determined by most likely path logic 440. The results of most likely path logic 440 may be evaluated by Viterbi scoring logic 442 to determine whether the reference mark alignment along the most likely path provides sufficient confidence that the reference mark timing can be accurately recovered. For example, Viterbi scoring logic 442 may sum the probabilities for each column without normalization to determine a confidence score for reference timing based on the selected reference pattern and corresponding reference matrix.

In some configurations, blocks 414-442 may be executed for each reference pattern in reference set 416 for the convolutional matrix from the read data. In some configurations, a reference set counter may be incremented to track progress through the reference set each time a next reference pattern from reference set 416 may be selected by next reference selector 414. The reference set counter may determine when confidence scores have been determined for each reference pattern using Viterbi scoring logic 442, and operation may proceed to score comparison logic 444. Score comparison logic 444 may compare the Viterbi scores for each reference pattern and corresponding reference matrix to determine the most likely reference pattern. For example, the reference pattern with the highest score or score closest to 1 may be selected as the most likely reference pattern. Reference determination logic 446 may receive an indicator of the most likely reference pattern and the corresponding Viterbi score. In some configurations, reference determination logic 446 may also include a confidence threshold for determining whether the most likely reference pattern is sufficiently reliable to use for further error correction and decoding. If that threshold is met, blocks 452 and 454 may use the selected reference pattern for oligo address determination and/or oligo copy sorting and/or blocks 458-468 may use the corresponding Viterbi soft information 450 from the correlation matrix for the selected reference pattern to execute insertion and/or deletion corrections to attempt to restore the original reference mark timing and/or other correlation-based base pair corrections.

In some configurations, reference marks and the reference patterns they include may encode the oligo index or oligo address. Once the most likely reference pattern is determined for the read data by reference determination logic 446, the oligo address may be determined from the reference pattern. Oligo address logic 454 may process the most likely reference pattern to determine the oligo address that it encodes, such as by reference patterns that are uniquely mapped to each oligo address in the oligo pool. In some configurations, this may include dividing the most likely reference pattern into a series of reference pattern segments, where each reference pattern segment maps to an oligo address segment value. The series of oligo address segment values may then be combined to recover the full oligo address. In some configurations, the oligo address may be encoded with one or more sets of redundancy data, and oligo address logic may perform an oligo address redundancy check to validate the oligo address or components thereof based on the oligo address data and corresponding redundancy data. Once the oligo address is successfully determined and validated, it may be used by oligo sorting logic 452 to sort copies of the oligo with the same oligo address into sets of multiple copies that can be used for consensus-based error correction using blocks 456-466 and/or populating a hyperblock by combining the oligo data from the set of oligos. Both oligo error correction and reassembly may include additional layers of error correction encoded in the oligo data.

The output of oligo sorting logic 452 may allow number of copies logic 456 to determine the number of copies of each oligo in the read data from the oligo pool. In some configurations, number of copies logic 456 may use the number of copies for each oligo to determine the processing path and consensus functions to be used for that particular oligo. For example, number of copies logic 456 may include one or more number of copy thresholds for determining the consensus processing. An oligo with only a single copy may not be eligible for consensus processing, but a number of copies greater than one may be able to use soft information consensus correction. In some configurations, the number of copies threshold may determine which processing path and corresponding consensus function is used for processing the soft information from the multiple copies. In some configurations, base pair aggregation logic 458 may aggregate soft information across multiple copies of the same oligo. For example, base pair aggregation logic 458 may generate base pair matrices similar to those shown in FIGS. 5C and 5D that aggregate and/or generate soft information based on Viterbi soft information 450 and/or hard decisions using consensus function 560. Consensus function (or functions) 460 may operate on the aggregated soft information and/or hard decisions through one or more functions that determine best guess values for the aggregate oligo data. Consensus function 460 may be applied to both reference mark and oligo payload data. The best guess reference positions may then be used by insertion/deletion correction 462 to delete insertions and replace deletions with placeholders to restore reference timing. In some configurations, the placeholders may be determined from base pair consensus across copies. The reference marks may be removed and the payload data and related soft information provided to oligo iterative decoder 464. Oligo iterative decoder 464 may use redundancy data and corresponding encoding within the oligo to decode the payload data. Aggregate oligo best copy logic 466 may return the best copy of the oligo payload base on the current set of copies for that oligo and aggregate oligo soft information 486 may return the corresponding soft information from consensus function 460 and/or oligo iterative decoder 464. These “best copies” and corresponding soft information for each oligo contributing to a hyperblock may then be provided to hyperblock assembly logic 470.

Hyperblock assembly logic 470 may receive and organize the current best copies of the payload data for each oligo. For example, as the best copy of each oligo is determined by aggregate best copy logic 466, the corresponding oligo address from oligo address logic 454 may be used to determine the sequential position of that set of payload data in the hyperblock. Hyperblock assembly logic 470 may aggregate the segments of oligo payload data in an ordered sequence in memory that forms an aggregate decode block for that set of oligos. In some configurations, hyperblock assembly logic 470 may store the hyperblock data to hyperblock storage 472. For example, hyperblock assembly logic 470 may sequentially write the segments corresponding to the sequence of oligo payloads in a buffer memory sized to receive the full hyperblock. Hyperblock iterative decoder 474 may operate on the hyperblock data to attempt to decode it using ECC codewords and redundancy data included in the original encoding of the hyperblock. For example, hyperblock iterative decoder 474 may use LDPC decoding to attempt to recover the original data encoded across the set of oligos. The decoded hyperblock may have an associated CRC value and hyperblock CRC 476 may execute a CRC check to determine whether the CRC condition is met. The CRC check may be used as a verification of successful decode (in addition to the convergence metrics of hyperblock iterative decoder 474) and may be used by hyperblock cycle logic 478 to determine whether additional copies and iterations are needed to successfully decode the hyperblock. For example, hyperblock cycle logic 478 may use the output of the CRC to determine whether the decode was successful or not. If the decode was successful, decoded hyperblock output 480 may be returned for the hyperblock and used to return the original data unit. If the decode was not successful, hyperblock cycle logic 476 may initiate additional amplification and sequencing through amplification/sequencing interface 482, such as an interface to an associated DNA sequencing system containing or operating on the oligo pool.

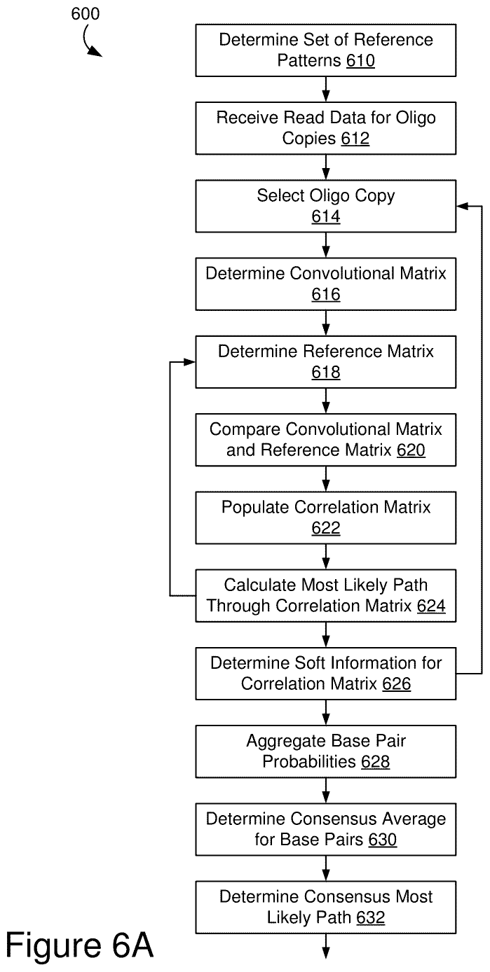

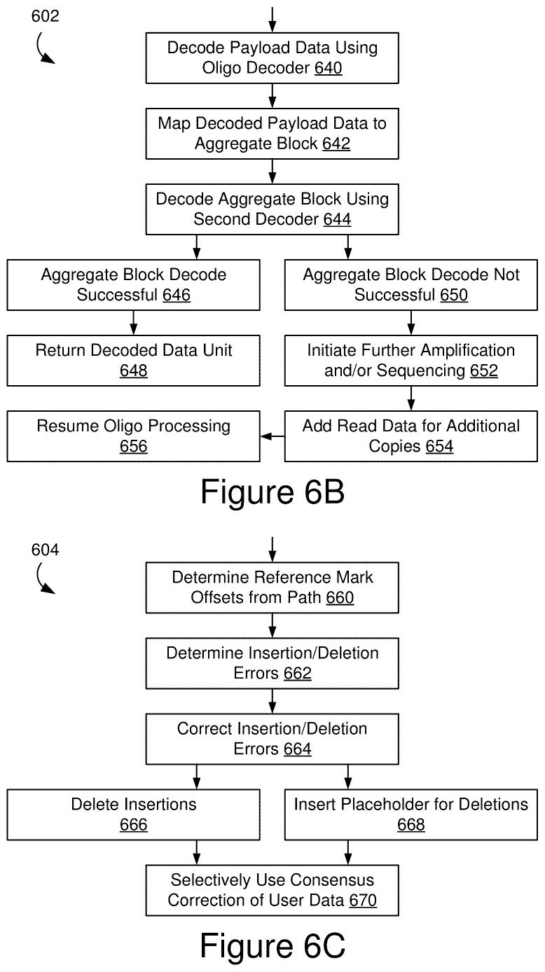

As shown in FIGS. 6A, 6B, and 6C, the decoder in decoding system 240 may be operated according to an example method of recovering reference timing from embedded reference marks in oligo read data, using consensus averaging of soft information for correcting insertion and deletion errors, and using decoding of aggregate hyperblocks to determine whether more oligo processing is needed i.e., according to the method 600 illustrated by blocks 610-670. In some configurations, sub-method 602 in FIG. 6B may selectively return the decoded data unit or initiate further amplification and sequencing for additional oligo copies. In some configurations, sub-method 604 may use the soft information consensus to establish reference mark locations and use them to correct insertion/deletion errors prior to proceeding to sub-method 602.

At block 610, a set of reference patterns may be determined. For example, the decoder may be configured with a set of reference patterns that include different sequences of reference mark values that correspond to one or more convolutional codes that are easily detected and statistically distinguishable from the oligo data.

At block 612, read data may be received for oligo copies from an oligo pool. For example, the decoder may receive sequences of base pairs corresponding to different oligos (including multiple copies of the same oligo) to be decoded.

At block 614, an oligo copy may be selected from the set of read data. For example, the read data from the oligo pool amplification and sequencing may include an unknown number of copies of each oligo that may be grouped into sets of oligo copies at one or more stages of data processing.

At block 616, a convolutional matrix may be determined. For example, the decoder may separate the reference data positions from the user data positions, organize them into paired columns, and then repeat those columns for a desired number of offsets in each direction, such as 5-6 single base pair offsets.

At block 618, a reference matrix may be determined for the oligo. For example, the decoder may repeat the base pairs from a selected reference pattern for each column of the reference matrix to match the number of columns in the convolutional matrix. In some configurations, the decoder may be configured to sequentially process each reference pattern from the set of reference patterns against the read data to determine the reference pattern that results in the best fit for the reference marks.

At block 620, the convolutional matrix may be compared to the reference matrix. For example, the decoder may use an exclusive-or to compare the base pair values in the convolutional matrix with the corresponding matrix position in the selected reference matrix.

At block 622, a correlation matrix may be populated. For example, the decoder may place a corresponding value from the comparisons at block 620 in the corresponding matrix position in a correlation matrix.

At block 624, a most likely path through the correlation matrix may be calculated or otherwise determined. For example, the decoder may apply a Viterbi algorithm to traverse the correlation matrix to determine relative probability values in each row of the correlation matrix for possible shifts in reference mark positions. In some configurations, a Viterbi score for each reference pattern and corresponding reference matrix may be calculated and compared for determining the reference matrix at block 618.

At block 626, soft information for the correlation matrix may be determined. For example, the decoder may use the probabilities generated by the Viterbi algorithm for each entry in the correlation matrix to determine a set of soft information for that oligo copy. If there are additional oligo copies to be evaluated, method 600 may return to block 614 to select a next oligo copy to be evaluated for reference mark recovery and error correction.

At block 628, base pair probabilities may be aggregated for each copy of the oligo. For example, the decoder may use the probabilities from each oligo copy's set of soft information to determine probability values for each possible base pair in each position of the oligo copy (reference positions and/or payload data positions).

At block 630, the consensus average for each base pair across oligo copies may be determined. For example, the decoder may use the base pair probabilities for each oligo copy determined at block 628 to execute one or more consensus functions targeting some or all positions in the set of oligo copies to determine a most likely base pair value for each position.

At block 632, a consensus most likely path may be determined. For example, the decoder may use the base pair values determined by consensus averaging at block 630 to determine the most likely path for the reference pattern, corresponding reference marks, and/or intervening payload data positions for further processing.

At block 640, payload data for each oligo may be decoded using an oligo decoder. For example, the decoder may process the best copy of the oligo payload data from the corrected reference timing and consensus functions through an ECC decoder based on the original oligo encoding.

At block 642, decoded payload data for each oligo may be mapped to an aggregate decode block. For example, each oligo payload may represent a segment of a hyperblock, and the decoder may determine the relative positions of the decoded oligo payload data to reassemble the aggregate data block for hyperblock decoding.

At block 644, the aggregate decode block may be decoded using a hyperblock decoder. For example, the decoder may process the hyperblock through an ECC decoder for the aggregate codeword size of the hyperblock.

At block 646, the decode of the aggregate decode block may be successful. For example, the decoder may determine that the hyperblock decoding was successful based on ECC metrics and/or a CRC check.

At block 648, the decoded data unit may be returned. For example, the decoder may return the decoded data from the hyperblock to recover the original data that was encoded in the oligo pool.

At block 650, the decode of the aggregate data block may not be successful. For example, the decoder may determine that the hyperblock decoding was unsuccessful based on ECC metrics and/or a CRC check.

At block 652, further amplification and/or sequencing may be initiated. For example, the decoder may generate a message to a DNA sequencing system to initiate additional sequencing from the oligo pool to generate additional copies of the oligos in read data.

At block 654, read data for additional copies may be added to the read data being used for recovering the hyperblock. For example, the decoder may receive additional read data corresponding to additional oligo sequencing from the same oligo pool to increase the number of copies used for reference timing and consensus correction.

At block 656, oligo processing may be resumed for the new read data. For example, the decoder may return to block 614 to process the additional copies and use them to improve the consensus most likely path determined at block 632 for each set of oligo copies.

At block 660, reference mark offsets may be determined from the consensus most likely path. For example, the decoder may interpret (relative to the most likely value in the prior row) shifts to the column on the left as one direction of offset and shifts to the column on the right as an opposite direction of offset.

At block 662, insertion and deletion errors may be determined from the offsets. For example, the decoder may evaluate the directions of the offsets as corresponding to sites of insertions or deletions in the oligo copy, such as left offsets corresponding to deletions and right offsets corresponding to insertions.

At block 664, insertion and deletion errors may be corrected in one or more of the oligo copies. For example, the decoder may include logic to insert placeholder values (which may be marked as erasures) for identified deletions at block 668 and delete base pairs identified as insertions at block 666 to compensate for errors and restore the reference mark timing of the user data segments between sequential reference marks.

At block 670, consensus correction of oligo payload or user data may be selectively used. For example, the decoder may use the soft information consensus averages of base pair values in the payload data positions where the soft information meets a consensus threshold prior to removing reference mark data and passing the resulting payload data for correction using error correction codes.

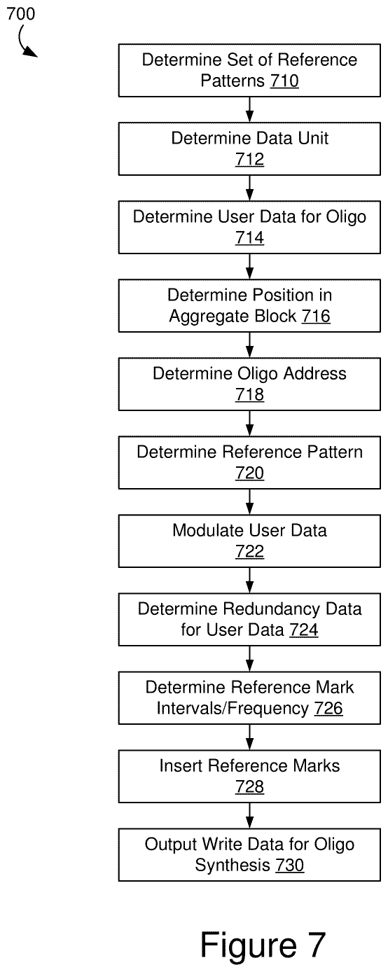

As shown in FIG. 7, the encoder in encoding system 210 may be operated according to an example method of encoding embedded reference marks that may include oligo index data in oligos, i.e., according to the method 700 illustrated by blocks 710-730.

At block 710, a set of reference patterns may be determined. For example, the encoder may be configured with a set of reference patterns that include different sequences of reference mark values that correspond to one or more convolutional codes that are easily detected and statistically distinguishable from the oligo data.

At block 712, a data unit may be determined. For example, the encoder may receive a data unit in a conventional binary data format for storage in a set of oligos.

At block 714, user data for the oligo may be determined. For example, an oligo formatter in the encoder may select a portion of user data from the data unit to be written to a target oligo.

At block 716, a position in an aggregate data block may be determined. For example, the encoder may determine a sequence of oligos that will include payload data for a hyperblock with aggregate ECC encoding.

At block 718, an oligo address may be determined for encoding the oligo. For example, the oligo formatter may determine an oligo address and/or other syntactic information to be included in the oligo to assist with recovery of the user data and mapping it into a larger user data structure or data unit (e.g., file or data object), as well as any hyperblock, that may also include encoding with redundancy data.

At block 720, a reference pattern for encoding reference marks in the oligo may be determined. For example, the encoder may include an index, data structure, or function for selecting reference patterns from a reference set and/or mapping the oligo address to corresponding reference patterns (e.g., distinct convolutional codes) from the set of reference patterns.

At block 722, user data may be modulated. For example, an RLL encoder or similar modulator may use a modulation code selected to assure that the user data does not include the selected reference mark code.

At block 724, redundancy data may be determined for the user data. For example, the user data may be encoded using an error correction code that generates corresponding redundancy data, such as parity data. Redundancy data may be generated for multiple levels of encoding, including oligo redundancy data and hyperblock redundancy data.

At block 726, reference mark intervals or frequency may be determined. For example, the reference mark formatter may be configured for a reference mark interval defining the number of base pairs that should appear between sequential reference marks in the oligo, which may be a single base pair for 0.5 reference mark frequency or code rate.

At block 728, reference marks may be inserted. For example, the reference mark inserter may insert the sequence of reference marks into the user data at the reference mark intervals to define a plurality of user data segments between each pair of sequential reference marks. The reference marks may correspond to the reference patterns selected at block 722 and ordered to form a complete reference pattern for all of the reference marks,

At block 730, write data for the oligo may be output for oligo synthesis. For example, the encoder may generate write data consisting of the user data segments for the target oligo and the reference marks (with embedded oligo index data) and any other added formatting data.

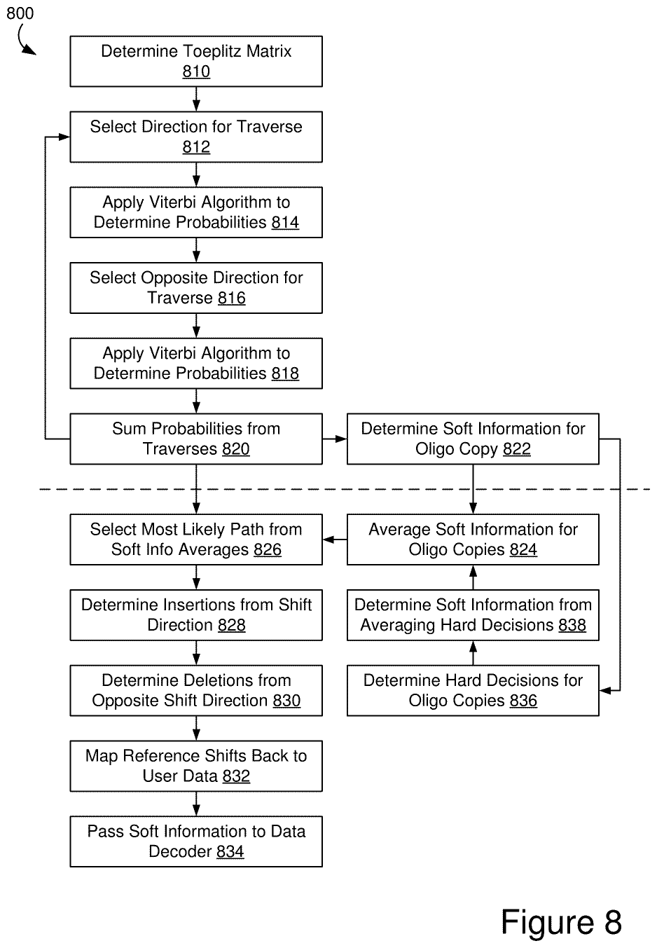

As shown in FIG. 8, the decoder in decoding system 240 and, more specifically, the reference mark decoder 250, may be operated according to an example method of determining probabilities of reference mark shifts using a Viterbi algorithm, i.e., according to the method 800 illustrated by blocks 810-838. In some configurations, the oligo data processing system 400 of FIG. 4 may be embodied at least in part in a reference mark decoder and/or data consensus correction decoder to execute method 800. In some configurations, method 800 may operate in the context of method 600 and, more specifically, provide further detail of the operations for blocks 624-632 on correlation matrices for generating soft information for multiple oligo copies.

At block 810, a Toeplitz matrix may be determined. For example, the decoder may include a Toeplitz matrix configured to modify probabilities for single base pair offsets from a prior reference offset position to determine column changes.

At block 812, a direction may be selected for traverse. For example, the decoder may be configured to traverse the correlation matrix in a first or alpha direction, such as from the top row or start of the read data sequence to the bottom row or end of the read data sequence.

At block 814, the Viterbi algorithm may be applied to determine probabilities for each position in the matrix based on the direction of traversal. For example, the decoder may calculate probabilities for each row through the correlation matrix based on the probability values calculated for the prior row in the correlation matrix based on the direction of the traversal.

At block 816, an opposite direction may be selected for traverse. For example, the decoder may be configured to traverse the correlation matrix in a second or beta direction, such as from the bottom row or end of the read data sequence to the top row or start of the read data sequence.

At block 818, the Viterbi algorithm may be applied to determine probabilities for each position in the matrix based on the direction of traversal. For example, the decoder may make the same calculations as at block 814, but in the opposite direction.

At block 820, probabilities from the traverses may be summed. For example, the decoder may combine the results for each position in the matrix across the two traverses in opposite directions, such as by adding, averaging, or variance calculation. In some configurations, operation may return to block 812 for a next copy of the oligo.

At block 822, soft information may be determined for each oligo copy. For example, the encoder may determine the matrix of probability values generated at blocks 814, and 818, and/or 820.

At block 624, the soft information from multiple oligo copies may be averaged. For example, the decoder may use one or more consensus functions based on the number of available copies to consensus average the likelihood of each possible base pair value for different oligo positions.

At block 826, a consensus most likely path may be selected from the consensus averaged soft information. For example, the decoder may evaluate the consensus averaged base pair values in each row of the matrix to determine which probability is the highest.

At block 828, insertions may be determined from shifts between adjacent rows in one direction. For example, the decoder may determine that column shifts between one row and the next (corresponding to adjacent reference mark positions) to the right correspond to insertion errors.

At block 830, deletions may be determined from shifts between adjacent rows in the opposite direction. For example, the decoder may determine that column shifts between one row and the next to the left correspond to deletion errors.

At block 832, reference mark shifts may be mapped back to user data. For example, the decoder may use the reference mark position shifts in the context of the full set of oligo positions (reference marks and user data) to locate insertions and deletions in the oligo read data in order to compensate for those shifts and correct reference mark positions/timing.

At block 834, the soft information may be passed to an iterative data decoder. For example, the decoder may pass the soft information to the iterative ECC decoder for the user data.

At block 836, hard decisions may be determined for oligo copies. For example, in addition to the soft information determined from the Viterbi algorithm, the decoder may generate hard decisions (most likely base pair value) for each base pair in the oligo.

At block 838, soft information may be determined from averaging hard decisions across oligo copies. For example, the decoder may generate additional soft information based on aggregating the hard decisions for each base pair across multiple copies of the oligo and this soft information may be used in the consensus functions used at block 826 to select a consensus most likely path.

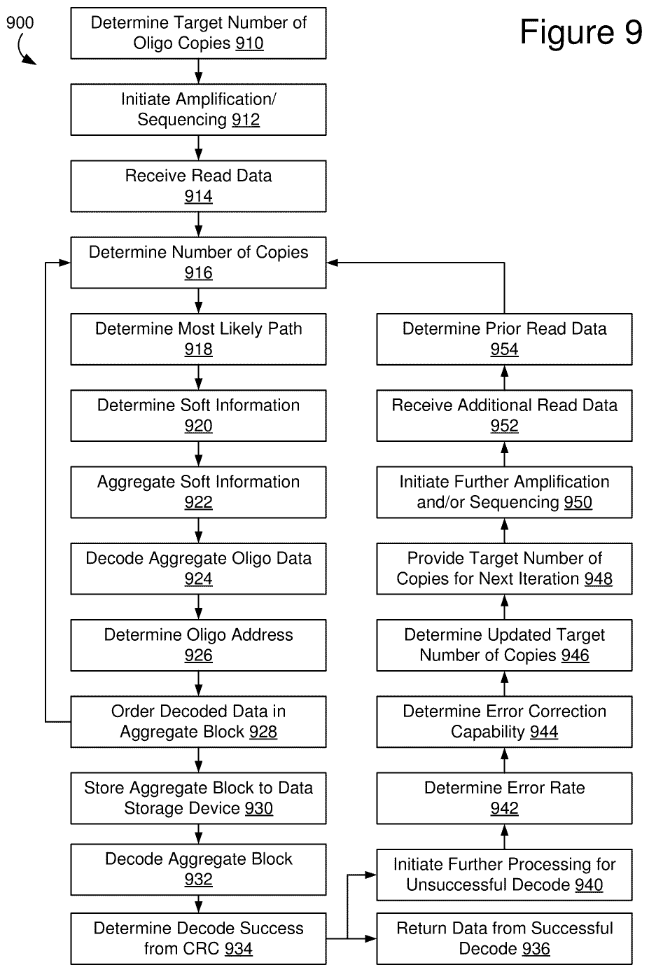

As shown in FIG. 9, the decoder in decoding system 240 and, more specifically, the hyperblock decoder logic 270 and surrounding components, may be operated according to an example method of using hyperblock decoding to iteratively decode an increasing number of oligo copies until the hyperblock can be successfully decoded, i.e., according to the method 900 illustrated by blocks 910-954.

At block 910, a target number of oligo copies may be determined. For example, the decoder may be configured for an initial or minimum number of copies that balance amplification and sequencing resources with a target likelihood of decoding in a single pass.

At block 912, amplification and sequencing are initiated for an oligo pool. For example, the decoder may send a message to a DNA sequencing system with a target oligo pool identifier and target number of copies based on the oligo pool's hyperblock encoding scheme, or the decoder may receive the first set of read data responsive to the DNA sequencing system by initiated by a user or another system.

At block 914, read data may be received. For example, the decoder may receive the first set of read data comprised of oligo data for a set of oligos and, ideally, including multiple copies of each oligo (though single copies and omissions may be possible).

At block 916, a number of copies may be determined. For example, the decoder may use statistical analysis or tagging to identify copies of the same oligo in the read data and group them and/or may use oligo address or other information within the oligo determined later in oligo processing to identify copies.

At block 918, a most likely path is determined for each oligo copy. For example, the decoder may use a correlation matrix and Viterbi algorithm to determine the most likely set of base pair values based on reference marks.

At block 920, soft information may be determined for each oligo copy. For example, the decoder may use probability values from the Viterbi algorithm to determine soft information for each base pair in the oligo copy.

At block 922, soft information may be aggregated for each oligo from multiple copies. For example, the decoder may aggregate the soft information values from block 920 across oligo copies for each base pair position into an aggregate set of soft information. In some configurations, aggregate soft information may be generated from aggregating hard decision information across the multiple copies.

At block 924, aggregate oligo data may be decoded. For example, the decoder may process a best copy of the oligo based on the aggregate soft information through an iterative decoder for the oligo data encoding.

At block 926, the oligo address may be determined. For example, the oligo address may indicate where the oligo data fits in the sequence of a hyperblock and the decoder may use the oligo address to map the decoded oligo data to positions in the hyperblock.

At block 928, decoded oligo data may be ordered in an aggregate decode block. For example, the decoder may order the decoded oligo data in the hyperblock based on oligo address.

At block 930, the aggregate decode block may be stored to a data storage device. For example, the decoder may write the decoded oligo data in sequence to a data storage device, such as a hard disk drive or soldi state drive.

At block 932, the aggregate decode block may be decoded. For example, the decoder may use an ECC iterative decoder for the hyperblock codeword size and encoding to attempt to decode the hyperblock.

At block 934, decode success may be determined from a CRC check. For example, the hyperblock may be encoded with a CRC value and the decoder may use a CRC check to verify whether the correct codeword was successfully decoded for the hyperblock.

At block 936, responsive to a successful decode, the decoded data from the aggregate decode block may be returned. For example, the decoder may return the decoded hyperblock data as user data or for further processing to determine a user data block based on the decoded hyperblock data.

At block 940, responsive to an unsuccessful decode, further processing may be initiated. For example, the decoder may include an iterative cycle logic for determining additional processing for additional copies to improve the likelihood of decoding the hyperblock. In some configurations, the decoder may include a threshold based on the number of iterations or other metrics for ending the iterations after an unsuccessful decode (declaring the hyperblock unrecoverable).

At block 942, an error rate may be determined. For example, the decoder may determine an error rate for the last unsuccessful hyperblock decoding operation based on the iterative decoder metrics and/or aggregate soft information for the oligos in the hyperblock.

At block 944, an error correction capability of the aggregate decode block decoder may be determined. For example, the hyperblock may be encoded with a particular ECC scheme with a known error correction capability and the decoder may map the encoding scheme to an error correction capability value.

At block 946, an updated target number of copies may be determined. For example, the decoder may compare the error rate from the prior read data to the error correction capability to determine an increased number of copies desired for a next iteration of the decoding process.

At block 948, a target number of copies for the next iteration of the sequencing may be provided. For example, the decoder may send a target number of copies based on the difference between the current number of copies and the updated target number of copies to increase the likelihood of decode. In some configurations, each iteration may be based on fixed increments of increasing target number of copies without relying on error rate or error correction capability analysis.

At block 950, further amplification and/or sequencing may be initiated. For example, the decoder may send a message to the DNA sequencing system to initiate additional sequencing (which may include additional amplification if all previously amplified oligos have already been sequenced) and may provide the target number of copies from block 948 to the DNA sequencing system to guide the magnitude of the additional sequencing.

At block 952, additional read data may be received. For example, the decoder may receive a next set of read data from the DNA sequencing system in response to the message for further sequencing.

At block 954, the prior read data may be determined. For example, the decoder may retain the read data from the previous iteration and combine it with the new read data to determine the number of copies and perform aggregate consensus processing for each oligo. Method 900 may not reprocess the oligo copies from the prior read data but combine the retained soft information and/or hard decisions for aggregating and decoding the aggregate oligo data during each successive iteration.

Technology for improved encoding and decoding of data for DNA storage is described above. In the above description, for purposes of explanation, numerous specific details were set forth. It will be apparent, however, that the disclosed technologies can be practiced without any given subset of these specific details. In other instances, structures and devices are shown in block diagram form. For example, the disclosed technologies are described in some implementations above with reference to particular hardware.

Reference in the specification to “one embodiment” or “an embodiment” means that a particular feature, structure, or characteristic described in connection with the embodiment is included in at least one embodiment or implementation of the disclosed technologies. The appearances of the phrase “in one embodiment” in various places in the specification are not necessarily all referring to the same embodiment or implementation.

Some portions of the detailed descriptions above may be presented in terms of processes and symbolic representations of operations on data bits within a computer memory. A process can generally be considered a self-consistent sequence of operations leading to a result. The operations may involve physical manipulations of physical quantities. These quantities take the form of electrical or magnetic signals capable of being stored, transferred, combined, compared, and otherwise manipulated. These signals may be referred to as being in the form of bits, values, elements, symbols, characters, terms, numbers, or the like.

These and similar terms can be associated with the appropriate physical quantities and can be considered labels applied to these quantities. Unless specifically stated otherwise as apparent from the prior discussion, it is appreciated that throughout the description, discussions utilizing terms for example “processing” or “computing” or “calculating” or “determining” or “displaying” or the like, may refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The disclosed technologies may also relate to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, for example, but is not limited to, any type of disk including floppy disks, optical disks, CD-ROMs, and magnetic disks, read-only memories (ROMs), random access memories (RAMs), erasable programmable read-only memories (EPROMs), electrically erasable programmable read-only memories (EEPROMs), magnetic or optical cards, flash memories including USB keys with non-volatile memory or any type of media suitable for storing electronic instructions, each coupled to a computer system bus.

The disclosed technologies can take the form of an entire hardware implementation, an entire software implementation or an implementation containing both hardware and software elements. In some implementations, the technology is implemented in software, which includes but is not limited to firmware, resident software, microcode, etc.

Furthermore, the disclosed technologies can take the form of a computer program product accessible from a non-transitory computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

A computing system or data processing system suitable for storing and/or executing program code will include at least one processor (e.g., a hardware processor) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modems, and Ethernet cards are just a few of the currently available types of network adapters.

The terms storage media, storage device, and data blocks are used interchangeably throughout the present disclosure to refer to the physical media upon which the data is stored.

Finally, the processes and displays presented herein may not be inherently related to any particular computer or other apparatus. Various general-purpose systems may be used with programs in accordance with the teachings herein, or it may prove convenient to construct more specialized apparatus to perform the required method operations. The required structure for a variety of these systems will appear from the description above. In addition, the disclosed technologies were not described with reference to any particular programming language. It will be appreciated that a variety of programming languages may be used to implement the teachings of the technologies as described herein.

The foregoing description of the implementations of the present techniques and technologies has been presented for the purposes of illustration and description. It is not intended to be exhaustive or to limit the present techniques and technologies to the precise form disclosed. Many modifications and variations are possible in light of the above teaching. It is intended that the scope of the present techniques and technologies be limited not by this detailed description. The present techniques and technologies may be implemented in other specific forms without departing from the spirit or essential characteristics thereof. Likewise, the particular naming and division of the modules, routines, features, attributes, methodologies and other aspects are not mandatory or significant, and the mechanisms that implement the present techniques and technologies or its features may have different names, divisions and/or formats. Furthermore, the modules, routines, features, attributes, methodologies and other aspects of the present technology can be implemented as software, hardware, firmware or any combination of the three. Also, wherever a component, an example of which is a module, is implemented as software, the component can be implemented as a standalone program, as part of a larger program, as a plurality of separate programs, as a statically or dynamically linked library, as a kernel loadable module, as a device driver, and/or in every and any other way known now or in the future in computer programming. Additionally, the present techniques and technologies are in no way limited to implementation in any specific programming language, or for any specific operating system or environment. Accordingly, the disclosure of the present techniques and technologies is intended to be illustrative, but not limiting.

1. A system, comprising:
at least one decoder circuit configured to, alone or in combination:
receive a first set of read data determined from sequencing a set of oligos that includes first numbers of copies of each oligo and corresponds to an oligo pool configured to store a data unit;
determine, from the first set of read data for each copy of each oligo, a correlation matrix of possible base pair offsets for that copy of the oligo;
determine, for each copy of each oligo, a most likely path through the correlation matrix for that copy of that oligo, wherein calculation of the most likely path generates a set of soft information for each element in the correlation matrix for that copy of that oligo;
aggregate, for each oligo, an aggregate set of soft information for each base pair along a length of that oligo;
decode, using a first iterative decoder, payload data in each oligo using the aggregate set of soft information for that oligo to determine decoded payload data for that oligo;
map decoded payload data for each oligo to data positions in an aggregate decoding block;
decode, using a second iterative decoder, the aggregate decoding block to determine a decoded aggregate decoding block for the data unit; and
determine, based on the decoded aggregate decoding block, whether the data unit can be successfully decoded.

2. The system of claim 1, wherein the at least one decoder circuit is further configured to, alone or in combination, responsive to determining that the data unit cannot be successfully decoded from the decoded aggregate decoding block:
selectively initiate further sequencing of the oligo pool to generate a next set of read data;
receive the next set of read data that includes a second number of copies of each oligo;
determine a prior set of read data based on the first set of read data and any subsequent iterations of selectively initiating further sequencing;
initiate, from the next set of read data and for each copy of each oligo, determining the correlation matrices, most likely paths, and sets of soft information from the next set of read data;
aggregate, for each oligo, an updated aggregate set of soft information using the sets of soft information from the prior set of read data and the next set of read data;
decode, using the first iterative decoder, the payload data in each oligo using the updated aggregate set of soft information for that oligo to determine updated decoded payload data for that oligo;
update the decoded payload data for each oligo in the aggregate decoding block;
decode, using the second iterative decoder, the aggregate decoding block to determine an updated decoded aggregate decoding block; and
determine, based on the updated decoded aggregate decoding block, whether the data unit can be successfully decoded.

3. The system of claim 2, wherein the at least one decoder circuit is further configured to, alone or in combination, iteratively execute, until the data unit is successfully decoded:
the selectively initiating further sequencing;
the receiving the next set of read data;
the determining the prior set of read data;
the initiating determining the correlation matrices, most likely paths, and sets of soft information;
the aggregating the updated aggregate set of soft information;
the decoding the payload data in each oligo;
the updating the decoded payload data for each oligo in the aggregate decoding block;
the decoding the aggregate decoding block; and
the determining whether the data unit can be successfully decoded.

4. The system of claim 2, wherein the at least one decoder circuit is further configured to, alone or in combination, responsive to determining that the data unit cannot be successfully decoded from the decoded aggregate decoding block:
determine a target number of copies of each oligo for selectively initiating further sequencing of the oligo pool to generate a next set of read data; and
provide the target number of copies to an amplification and sequencing system for the oligo pool.

5. The system of claim 4, wherein:
the at least one decoder circuit is further configured to, alone or in combination, responsive to determining that the data unit cannot be successfully decoded from the decoded aggregate decoding block:
determine an error rate for the first set of read data; and
determine an error correction capability for the second iterative decoder; and
determining the target number of copies of each oligo is based on the error rate for the first set of read data and the error correction capability for the second iterative decoder.

6. The system of claim 1, wherein the at least one decoder circuit is further configured to, alone or in combination, prior to decoding the payload data in each oligo:
determine, for each oligo, most likely positions for a series of reference marks configured with a reference pattern and a predetermined interval of base pairs;
correct, for each oligo, insertions and deletions to restore the predetermined interval of base pairs for the series of reference marks; and
remove, for each oligo, the reference marks from remaining base pairs in that oligo to determine the payload data to be decoded.

7. The system of claim 6, wherein the at least one decoder circuit is further configured to, alone or in combination:
determine, from the first set of read data for each copy of each oligo, a convolutional matrix for that copy of that oligo, wherein each column of the convolutional matrix corresponds to a base pair offset of the read data for that copy of the oligo;
determine, for each copy of each oligo, at least one reference matrix based on at least one possible reference pattern for that oligo; and
compare, for each copy of each oligo, the convolutional matrix to the at least one reference matrix to determine the correlation matrix of possible base pair offsets for that copy of the oligo.

8. The system of claim 1, further comprising:
a data storage device configured with a read channel block size, wherein:
the at least one decoder circuit is further configured to, alone or in combination, store, prior to decoding the aggregate decoding block, the aggregate decoding block to a non-volatile storage medium of the data storage device; and
the read channel block size is at least as large as a block size of the aggregate decoding block.

9. The system of claim 1, wherein mapping the decoded payload data for each oligo to data positions in the aggregate decoding block comprises:
determining, for each oligo in the set of oligos, an oligo address for that oligo configured to indicate a position of the decoded payload data of that oligo relative to positions of the decoded payload data of other oligos; and
ordering, based on the oligo addresses for each oligo in the set of oligos, the decoded payload data in the aggregate decoding block.

10. The system of claim 1, wherein determining whether the data unit can be successfully decoded comprises:
executing a cyclic redundancy check on the decoded aggregate decoding block;
outputting, responsive to the cyclic redundancy check being successful, the decoded aggregate decoding block for recovering and storing the data unit; and
initiating, responsive to the cyclic redundancy check not being successful, further amplification and replication of the oligo pool.

11. A method comprising:
receiving a first set of read data determined from sequencing a set of oligos that includes first numbers of copies of each oligo and corresponds to an oligo pool configured to store a data unit;
determining, from the first set of read data for each copy of each oligo, a correlation matrix of possible base pair offsets for that copy of the oligo;
determining, for each copy of each oligo, a most likely path through the correlation matrix for that copy of that oligo, wherein calculation of the most likely path generates a set of soft information for each element in the correlation matrix for that copy of that oligo;
aggregating, for each oligo, an aggregate set of soft information for each base pair along a length of that oligo;
decoding, using a first iterative decoder, payload data in each oligo using the aggregate set of soft information for that oligo to determine decoded payload data for that oligo;
mapping decoded payload data for each oligo to data positions in an aggregate decoding block;
decoding, using a second iterative decoder, the aggregate decoding block to determine a decoded aggregate decoding block for the data unit; and
determining, based on the decoded aggregate decoding block, whether the data unit can be successfully decoded.

12. The method of claim 11, further comprising, responsive to determining that the data unit cannot be successfully decoded from the decoded aggregate decoding block:
selectively initiating further sequencing of the oligo pool to generate a next set of read data;
receiving the next set of read data that includes a second number of copies of each oligo;
determining a prior set of read data based on the first set of read data and any subsequent iterations of selectively initiating further sequencing;
initiating, from the next set of read data and for each copy of each oligo, determining the correlation matrices, most likely paths, and sets of soft information from the next set of read data;
aggregating, for each oligo, an updated aggregate set of soft information using the sets of soft information from the prior set of read data and the next set of read data;
decoding, using the first iterative decoder, the payload data in each oligo using the updated aggregate set of soft information for that oligo to determine updated decoded payload data for that oligo;
updating the decoded payload data for each oligo in the aggregate decoding block;
decoding, using the second iterative decoder, the aggregate decoding block to determine an updated decoded aggregate decoding block; and
determining, based on the updated decoded aggregate decoding block, whether the data unit can be successfully decoded.

13. The method of claim 12, further comprising:
iteratively executing, until the data unit is successfully decoded:
the selectively initiating further sequencing;
the receiving the next set of read data;
the determining the prior set of read data;
the initiating determining the correlation matrices, most likely paths, and sets of soft information;
the aggregating the updated aggregate set of soft information;
the decoding the payload data in each oligo;
the updating the decoded payload data for each oligo in the aggregate decoding block;
the decoding the aggregate decoding block; and
the determining whether the data unit can be successfully decoded.

14. The method of claim 12, further comprising, responsive to determining that the data unit cannot be successfully decoded from the decoded aggregate decoding block:
determining a target number of copies of each oligo for selectively initiating further sequencing of the oligo pool to generate a next set of read data; and
providing the target number of copies to an amplification and sequencing system for the oligo pool.

15. The method of claim 14, further comprising, responsive to determining that the data unit cannot be successfully decoded from the decoded aggregate decoding block:
determining an error rate for the first set of read data; and
determining an error correction capability for the second iterative decoder, wherein determining the target number of copies of each oligo is based on the error rate for the first set of read data and the error correction capability for the second iterative decoder.

16. The method of claim 11, further comprising, prior to decoding the payload data in each oligo:
determining, for each oligo, most likely positions for a series of reference marks configured with a reference pattern and a predetermined interval of base pairs;
correcting, for each oligo, insertions and deletions to restore the predetermined interval of base pairs for the series of reference marks; and
removing, for each oligo, the reference marks from remaining base pairs in that oligo to determine the payload data to be decoded.

17. The method of claim 16, further comprising:
determining, from the first set of read data for each copy of each oligo, a convolutional matrix for that copy of that oligo, wherein each column of the convolutional matrix corresponds to a base pair offset of the read data for that copy of the oligo;
determining, for each copy of each oligo, at least one reference matrix based on at least one possible reference pattern for that oligo; and
comparing, for each copy of each oligo, the convolutional matrix to the at least one reference matrix to determine the correlation matrix of possible base pair offsets for that copy of the oligo.

18. The method of claim 11, wherein mapping the decoded payload data for each oligo to data positions in the aggregate decoding block comprises:
determining, for each oligo in the set of oligos, an oligo address for that oligo configured to indicate a position of the decoded payload data of that oligo relative to positions of the decoded payload data of other oligos; and
ordering, based on the oligo addresses for each oligo in the set of oligos, the decoded payload data in the aggregate decoding block.

19. The method of claim 11, wherein determining whether the data unit can be successfully decoded comprises:
executing a cyclic redundancy check on the decoded aggregate decoding block;
outputting, responsive to the cyclic redundancy check being successful, the decoded aggregate decoding block for recovering and storing the data unit; and
initiating, responsive to the cyclic redundancy check not being successful, further amplification and replication of the oligo pool.

20. A system comprising:
at least one processor;
at least one memory;
means for receiving a first set of read data determined from sequencing a set of oligos that includes first numbers of copies of each oligo and corresponds to an oligo pool configured to store a data unit;
means for determining, from the first set of read data for each copy of each oligo, a correlation matrix of possible base pair offsets for that copy of the oligo;
means for determining, for each copy of each oligo, a most likely path through the correlation matrix for that copy of that oligo, wherein calculation of the most likely path generates a set of soft information for each element in the correlation matrix for that copy of that oligo;
means for aggregating, for each oligo, an aggregate set of soft information for each base pair along a length of that oligo;
means for decoding, using a first iterative decoder, payload data in each oligo using the aggregate set of soft information for that oligo to determine decoded payload data for that oligo;
means for mapping decoded payload data for each oligo to data positions in an aggregate decoding block;
means for decoding, using a second iterative decoder, the aggregate decoding block to determine a decoded aggregate decoding block for the data unit; and
means for determining, based on the decoded aggregate decoding block, whether the data unit can be successfully decoded.
